SEQUENCE LISTING (29 sequences)

SEQ ID NO: 1 - protein, 4 aa, Artificial sequence
  synthetic peptide motif
CGPC

SEQ ID NO: 2 - protein, 105 aa, Artificial sequence
  synthetic peptide motif
  misc_feature (35)..(35): Xaa = any amino acid other than cysteine
MVKQIESKTAFQEALDAAGDKLVVVDFSATWCGPXKMIKPFFHSLSEKYSNVIFLEVDVDDCQDVASECEVKCMPTFQFFKKGQKVGEFSGANKEKLEATINELV

SEQ ID NO: 3 - protein, 6 aa, Artificial sequence
  synthetic peptide motif
WCGPCK

SEQ ID NO: 4 - protein, 109 aa, Pseudomonas syringae
MSNDLIKHVTDASFEADVLKADGAVLVDYWAEWCGPCKMIAPVLDEIATTYAGKLTIAKLNIDENQETPAKHGVRGIPTLMLFKNGNVEATKVGALSKSQLAAFLDANI

SEQ ID NO: 5 - protein, 104 aa, Porphyromonas gingivalis
MALQITDATFDGLVAEGKPMVVDFWATWCGPCRMVGPIIDELAAEYEGRAIIGKVDVDANTELPMKYGVRNIPTILFIKNGEVVKKLVGAQSKDVFKKELDALF

SEQ ID NO: 6 - protein, 103 aa, Listeria monocytogenes
MVKEITDATFEQETSEGLVLTDFWATWCGPCRMVAPVLEEIQEERGEALKIVKMDVDENPETPGSFGVMSIPTLLIKKDGEVVETIIGYRPKEELDEVINKYV

SEQ ID NO: 7 - protein, 103 aa, Saccharomyces cerevisiae
MVTQFKTASEFDSAIAQDKLVVVDFYATWCGPCKMIAPMIEKFSEQYPQADFYKLDVDELGDVAQKNEVSAMPTLLLFKNGKEVAKVVGANPAAIKQAIAANA

SEQ ID NO: 8 - protein, 105 aa, Gallus gallus
MVKSVGNLADFEAELKAAGEKLVVVDFSATWCGPCKMIKPFFHSLCDKFGDVVFIEIDVDDAQDVATHCDVKCMPTFQFYKNGKKVQEFSGANKEKLEETIKSLV

SEQ ID NO: 9 - protein, 105 aa, Mus musculus
MVKLIESKEAFQEALAAAGDKLVVVDFSATWCGPCKMIKPFFHSLCDKYSNVVFLEVDVDDCQDVAADCEVKCMPTFQFYKKGQKVGEFSGANKEKLEASITEYA

SEQ ID NO: 10 - protein, 105 aa, Rattus norvegicus
MVKLIESKEAFQEALAAAGDKLVVVDFSATWCGPCKMIKPFFHSLCDKYSNVVFLEVDVDDCQDVAADCEVKCMPTFQFYKKGQKVGEFSGANKEKLEATITEFA

SEQ ID NO: 11 - protein, 105 aa, Bos taurus
MVKQIESKYAFQEALNSAGEKLVVVDFSATWCGPCKMIKPFFHSLSEKYSNVVFLEVDVDDCQDVAAECEVKCMPTFQFFKKGQKVGEFSGANKEKLEATINELI

SEQ ID NO: 12 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 13 - protein, 134 aa, Arabidopsis thaliana
MGGALSTVFGSGEDAAAAGTESSEPSRVLKFSSSARWQLHFNEIKESNKLLVVDFSASWCGPCRMIEPAIHAMADKFNDVDFVKLDVDELPDVAKEFNVTAMPTFVLVKRGKEIERIIGAKKDELEKKVSKLRA

SEQ ID NO: 14 - protein, 167 aa, Zea mays
MAMETCFRAWALHAPAGSKDRLLVGNLVLPSKRALAPLSVGRVATRRPRHVCQSKNAVDEVVVADEKNWDGLVMACETPVLVEFWAPWCGPCRMIAPVIDELAKDYAGKITCCKVNTDDSPNVASTYGIRSIPTVLIFKGGEKKESVIGAVPKSTLTTLIDKYIGSS

SEQ ID NO: 15 - protein, 172 aa, Oryza sativa
MALETCFRAWATLHAPQPPSSGGSRDRLLLSGAGSSQSKPRLSVASPSPLRPASRFACQCSNVVDEVVVADEKNWDSMVLGSEAPVLVEFWAPWCGPCRMIAPVIDELAKEYVGKIKCCKVNTDDSPNIATNYGIRSIPTVLMFKNGEKKESVIGAVPKTTLATIIDKYVSS

SEQ ID NO: 16 - protein, 4 aa, Artificial Sequence
  synthetic peptide motif
  MISC_FEATURE (2)..(3): Xaa = any amino acid other than cysteine
CXXC

SEQ ID NO: 17 - protein, 4 aa, Artificial Sequence
  synthetic peptide motif
  MISC_FEATURE (2)..(4): Xaa = any amino acid other than cysteine
CXXX

SEQ ID NO: 18 - protein, 4 aa, Artificial Sequence
  synthetic peptide motif
  MISC_FEATURE (4)..(4): Xaa = any amino acid other than cysteine
CGPX

SEQ ID NO: 19 - protein, 6 aa, Artificial Sequence
  synthetic peptide motif
  MISC_FEATURE (1)..(1): Xaa = any amino acid other than cysteine
  MISC_FEATURE (3)..(6): Xaa = any amino acid other than cysteine
XCXXXX

SEQ ID NO: 20 - protein, 6 aa, Artificial Sequence
  synthetic peptide motif
  MISC_FEATURE (1)..(1): Xaa = any amino acid other than cysteine
  MISC_FEATURE (3)..(4): Xaa = any amino acid other than cysteine
  MISC_FEATURE (6)..(6): Xaa = any amino acid other than cysteine
XCXXCX

SEQ ID NO: 21 - protein, 6 aa, Artificial Sequence
  synthetic peptide motif
  MISC_FEATURE (1)..(1): Xaa = any amino acid other than cysteine
  MISC_FEATURE (5)..(6): Xaa = any amino acid other than cysteine
XCGPXX

SEQ ID NO: 22 - protein, 6 aa, Artificial Sequence
  synthetic peptide motif
  MISC_FEATURE (1)..(1): Xaa = any amino acid other than cysteine
  MISC_FEATURE (6)..(6): Xaa = any amino acid other than cysteine
XCGPCX

SEQ ID NO: 23 - protein, 6 aa, Artificial Sequence
  synthetic peptide motif
  MISC_FEATURE (5)..(5): Xaa = any amino acid other than cysteine
WCGPXK

SEQ ID NO: 24 - protein, 4 aa, Artificial Sequence
  synthetic peptide motif
  MISC_FEATURE (2)..(3): Xaa = any amino acid other than cysteine
CXXS

SEQ ID NO: 25 - protein, 6 aa, Artificial Sequence
  synthetic peptide motif
  MISC_FEATURE (1)..(1): Xaa = any amino acid other than cysteine
  MISC_FEATURE (3)..(4): Xaa = any amino acid other than cysteine
  MISC_FEATURE (6)..(6): Xaa = any amino acid other than cysteine
XCXXSX

SEQ ID NO: 26 - protein, 6 aa, Artificial Sequence
  synthetic peptide motif
  MISC_FEATURE (1)..(1): Xaa = any amino acid other than cysteine
  MISC_FEATURE (6)..(6): Xaa = any amino acid other than cysteine
XCGPSX

SEQ ID NO: 27 - protein, 6 aa, Artificial Sequence
  synthetic peptide motif
WCGPSK

SEQ ID NO: 28 - protein, 105 aa, Artificial Sequence
  synthetic peptide motif
  MISC_FEATURE (35)..(35): Xaa is any amino acid other than cysteine
  MISC_FEATURE (62)..(62): Xaa is any amino acid other than cysteine
  MISC_FEATURE (69)..(69): Xaa is any amino acid other than cysteine
  MISC_FEATURE (73)..(73): Xaa is any amino acid other than cysteine
MVKQIESKTAFQEALDAAGDKLVVVDFSATWCGPXKMIKPFFHSLSEKYSNVIFLEVDVDDXQDVASEXEVKXMPTFQFFKKGQKVGEFSGANKEKLEATINELV

SEQ ID NO: 29 - protein, 105 aa, Artificial Sequence
  synthetic peptide motif
MVKQIESKTAFQEALDAAGDKLVVVDFSATWCGPSKMIKPFFHSLSEKYSNVIFLEVDVDDSQDVASESEVKSMPTFQFFKKGQKVGEFSGANKEKLEATINELV

(19) United States
(12) Patent Application Publication
(10) Pub. No.: US 2023/0071765 A1
(43) Pub. Date: Mar. 9, 2023

(54) COMPOSITIONS HAVING THIOREDOXIN ACTIVITY AND RELATED METHODS

(71) Applicants: Peter B. HEIFETZ, San Diego, CA (US); Haim MOSKOWITZ, San Diego, CA (US); ORPRO THERAPEUTICS, INC., San Diego, CA (US)

(72) Inventors: Peter B. Heifetz, San Diego, CA (US); Haim Moskowitz, San Diego, CA (US)

(21) Appl. No.: 17/758,352

(22) PCT Filed: Jan. 4, 2021
(86) PCT No.: PCT/US2021/012109

§ 371 Date: Jul. 1, 2022

Related U.S. Application Data
(60) Provisional application No. 62/956,994, filed on Jan. 3, 2020.

(51) Int. Cl. C07K 4/00; A61K 9/14; A61K 38/03; A61P 29/00

(52) U.S. Cl. CPC C07K 4/00; A61K 9/14; A61K 38/03; A61P 29/00

(57) ABSTRACT

The present disclosure relates to preparations, formulations and uses of a protein or peptide having thioredoxin action for treating diseases and/or conditions. One aspect of the invention is a method to decrease viscoelasticity of mucus or sputum in a patient that has excessively viscous or cohesive mucus or sputum. The method includes contacting the mucus or sputum of the patient with a composition comprising a protein or peptide comprising a thioredoxin monocysteinic active site, wherein the protein or peptide does not contain any cysteine residues except for a single Cys at the N-terminal position of the thioredoxin monocysteinic active site.

### CROSS-REFERENCE TO RELATED APPLICATION

This application claims the benefit of U.S. Provisional Patent Application 62/956,994, filed Jan. 3, 2020, the entirety of which is incorporated herein by reference.

### REFERENCE TO SEQUENCE LISTING

This application contains a Sequence Listing submitted electronically as a text file by EFS-Web. The text file, named “7579-2-PROV_sequence_listing_v2_ST25.txt”, has a size in bytes of 21 KB, and was recorded on Jan. 2, 2020. The information contained in the text file is incorporated herein by reference in its entirety pursuant to 37 CFR § 1.52(e)(5).

### FIELD OF THE INVENTION

This invention relates generally to the preparation, formulation and use of a thioredoxin protein or peptide containing a thioredoxin active site in a reduced state for treating diseases and/or conditions such as reducing viscoelasticity of mucus or sputum, inflammation and hypertension.

### BACKGROUND OF THE INVENTION

Thioredoxin (Trx) is an essential intracellular human gene product that is also secreted on mucosal epithelia of the lung, upper and lower GI, eye and reproductive tract where together with the small tripeptide glutathione (GSH) it comprises the majority of extracellular biological reducing power. In contrast to intracellular proteins where most cysteine (Cys) residues are kept reduced by the local reducing environment, oxygen exposure causes most Cys of extracellular proteins to form covalent disulfide bonds. Biological reductants act to reverse this disulfide bonding, non-selectively in the case of GSH and other small-molecule thiols, but selectively in the case of thioredoxin oxidoreductases whose structural and chemical features confer specificity for only certain disulfide conformations. Secreted thioredoxin has evolved to serve a range of homeostatic functions via a unique and efficient thiol-disulfide exchange mechanism that targets protein disulfides having specific conformations including allosteric bond configurations associated with reversible regulatory control by thioredoxin-family oxidoreductases, and vicinal and other highly-constrained disulfides such as those formed intramolecularly in oxidized mucus proteins (mucins) where reduction by thioredoxin results in significant viscoelasticity normalization of CF patient sputum. Secreted thioredoxin exerts a range of anti-inflammatory effects via regulation of mediator release and inhibition of neutrophil chemotaxis to inflammatory sites (Tian, H., Matsuo, Y., Fukunaga, A., Ono, R., Nishigori, C., and Yodoi, J., 2013, Thioredoxin ameliorates cutaneous inflammation by regulating the epithelial production and release of pro-inflammatory cytokines. Frontiers in Immunology 4: 1-12), as well as inhibition of pro-inflammatory protease activity (Lee, R. L., Rancourt, R. C., del Val, G., Pack, K., Pardee, C., Accurso, F. J., and White, C. W., 2005, Thioredoxin and dihydrolipoic acid inhibit elastase activity in cystic fibrosis sputum. Am J Physiol Lung Cell Mol Physiol 289: L875-882). Thioredoxin has also been identified as the specific extracellular activator of a class of constitutively-expressed endogenous anti-microbial proteins (defensins) which are secreted on mucosal surfaces and exhibit markedly enhanced potency and target pathogen range upon thioredoxin-mediated reduction of their central disulfide bonds (Jaeger et al., 2013, Cell-mediated reduction of human β-defensin 1: a major role for mucosal thioredoxin. Mucosal immunology 6, 1179-90). Thioredoxin is furthermore classically considered an antioxidant protein due to selective activation of peroxidases and ability to directly donate electrons to certain oxidized substrates.

A large unmet medical need exists for safe, well-tolerated and effective drugs for the treatment of patients with diseases characterized by thickened, pathologic mucus, chronic infection, and chronic inflammation. One such disease is cystic fibrosis (CF) a genetic disorder resulting from mutation of the gene encoding the Cystic Fibrosis Transmembrane Regulator, CFTR, a key trans-membrane channel responsible for maintaining normal epithelial transport of chloride and bicarbonate ions. Defects in expression, accumulation or function of CFTR arising from nearly 2000 cftr gene mutations decrease cAMP-mediated release of chloride and transport of bicarbonate, leading to dehydration and increased viscoelasticity of airway mucus, decrease in periciliary layer depth and impaired mucociliary transport (MCT). Accumulation of the resulting poorly-cleared, pathologic mucus in the airways is central to the development of the chronic endobronchial bacterial infection and perpetual neutrophilic inflammation characteristic of CF (Fahy, J. V., and Dickey, B. F., 2010, Airway Mucus Function and Dysfunction. NEJM 363: 2233-2247). CF remains the most common inherited lethal disease in populations of primarily Northern European descent, affecting more than 30,000 individuals in the United States and over 80,000 worldwide. Chronic cough, excessive sputum production and respiratory complications are the principal causes of morbidity and decreased quality of life. While the life expectancy of CF patients has continued to increase, from 18 years prior to 1980 to over 40 years today, there is still an urgent need for improved therapies to further extend life expectancy and enhance quality of life. Therapies that are independent of CF genotype are particularly desirable.

Mucus is a continuously-secreted supramolecular polymer gel that forms a protective barrier on epithelial surfaces and is responsible via ciliary action and cough for transporting inhaled debris and bacteria out of the lung. Proper viscoelasticity and hydration of the mucus layer, which enables efficient cilia-driven transport is therefore critical to mucus function and the prevention of infection and inflammation. Normal mucus consists of mostly water (97%) with the remaining solids comprising mucin proteins, non-mucin proteins, salts, lipids and cellular debris. The polymeric mucin glycoproteins MUC5AC and MUC5B are primarily responsible for the viscoelastic properties of the respiratory mucus gel. O-linked glycan hydroxyl groups contribute water-binding, while the mucins themselves form an entangled network that also involves covalent and non-covalent interchain and intrachain linkages. The polymeric mucins are hyper-secreted in response to disease stress and inflammation and are remarkable for their extraordinarily high cysteine content—294 and 273 Cys per mature monomer for MUC5AC (UniProt accession P98088) and MUC5B (UniProt accession Q9HC84), respectively. These abundant mucin Cys have the potential to form numerous intrachain disulfides when exposed to 02 in the airway, with nearly a seven-fold increase in mucin disulfide bonding observed in CF patients vs. normal individuals (Yuan et al., 2015, Oxidation increases mucin polymer cross-links to stiffen airway mucus gels, Science Translational Medicine 7, 276ra227).

Analogous to the shortening of tightly-wound rubber bands, increased intrachain disulfide bonding in pathologic mucus gels contracts mucin filaments and compacts the polymeric mucus gel structure. This disulfide-mediated tightening of the CF mucin mesh may provide a mechanism for the observed increase in mucus concentration and osmotic modulus implicated in causing dehydration of the periciliary layer (PCL) and loss of mucus transport present in individuals affected by CF. The fundamental importance in CF pathophysiology of increased mucus viscoelasticity rather than dehydration per se is supported by direct measurement of PCL hydration and MCT on the epithelial surface of living airways using recently-developed, high-resolution noninvasive imaging techniques (Birket, S. E., et al., 2014, A functional anatomic defect of the cystic fibrosis airway, American Journal of Respiratory and Critical Care Medicine 190, 421-432; Chu, K. K., et al., 2016, In vivo imaging of airway cilia and mucus clearance with micro-optical coherence tomography, Biomed Opt Express 7, 2494-2505).

Naturally-secreted GSH and thioredoxins are likely the compounds responsible for preventing excess mucin disulfide bond formation in extracellular mucus. Increased net mucosal surface disulfide bonding will result from either reductant deficiency or elevated mucin protein levels, given the equilibrium between oxidation-driven disulfide formation and reductant-driven disulfide disruption. Mucus is known to be hyper-secreted in CF, and it has been observed that there is a ˜70% decrease in both reduced and oxidized forms of glutathione in CF patients compared to normal subjects (Wetmore, D. R., et al., 2010, Metabolomic profiling reveals biochemical pathways and biomarkers associated with pathogenesis in cystic fibrosis cells. JBC 285: 30516-22). This GSH decrease is consistent across multiple published studies investigating extracellular lung fluids of CF patients suggesting a role, likely indirect, for functional CFTR in maintaining normal rates of airway GSH efflux.

Even more significantly, impaired CFTR-mediated bicarbonate efflux in the CF epithelia is associated with in an abnormally acidic airway pH which decreases from 7.2 in normal individuals to less than 6.5 in CF patients (Garland A L, Walton W G, Coakley R D, et al., 2013, Molecular basis for pH-dependent mucosal dehydration in cystic fibrosis airways, Proceedings of the National Academy of Sciences 110:15973-8).

Because of the inherently high acid-dissociation constants (pKa) of small-molecule thiol agents, a low pH environment greatly attenuates the ability of endogenous or exogenous GSH (pKa 9.1) to form the deprotonated, reactive free thiolate anions necessary for nucleophilic disulfide bond attack. Only 0.25% of the natural GSH pool is calculated to be in the active, thiolate form at CF airway pH. Thus, not only is there an increase in secreted mucus (and hence mucin Cys capable of forming disulfide bonds) in CF and a decrease in GSH secretion, the GSH which remains is functionally impaired due to the reduced pH of the CF airway.

Likewise, related thiol compounds used as investigational or approved mucolytic agents including NAC, cysteamine, and Mesna (pKa values of 9.5, 8.3 and 9.2, respectively) similarly lack the potential for significant disulfide reducing activity in diseased airways. Moreover, these agents derive their mechanism from simple reduced thiols that promiscuously target any oxidized substrate without the selectivity characteristic of enzyme therapeutics.

In contrast, thioredoxin, a large molecule with an unusually acidic pKa of 6.2 resulting from hydrogen-bonding in its highly conserved enzyme active site, is dramatically less sensitive to acidic pH. Recent proteomic studies have revealed that thioredoxins comprise a significant proportion of the submucosal gland proteins that are secreted along with newly-formed airway mucus (Joo, N. S., Evans, I. A., Cho, H. J., Park, I. H., Engelhardt, J. F., and Wine, J. J. 2015. Proteomic analysis of pure human airway gland mucus reveals a large component of protective proteins. PLoS One 10, e0116756), suggesting a more significant functional role for this redox enzyme in airway disulfide bond homeostasis than has previously been considered.

Treating pathological mucus: Therapeutically, clearance of mucus from obstructed airways is a key aspect of mitigating ongoing chronic infection and inflammation in obstructive/inflammatory diseases like CF. Physical therapy, mechanical percussion devices and inhaled mucus-liquefying (mucolytic) medications are all components of the current treatment regimen for dislodgement of sputum in CF patients. However, existing treatments are largely symptomatic and have not been shown to be effective in mitigating the underlying mucus defects that lead mechanistically to poor clearance.

The most commonly used mucolytic compound in CF is recombinant human DNase I (DNase; Dornase Alfa), trademarked as Pulmozyme® by Genentech. DNase improves lung function by hydrolyzing viscous, accumulated neutrophil-derived nucleic acids, although recent research has shown that excess disulfide bonding in mucus proteins, rather than extracellular DNA accumulation, may play the dominant role in disease development.

Despite its broad usage, DNase has many disadvantages. DNase is a disulfide-bonded and glycosylated human enzyme of moderately large monomer size, which requires mammalian cell culture for manufacture, making it one of the more costly types of drugs to produce. The target of DNase, excess free nucleic acid, is present as a consequence of severe and chronic infection and might not be found at appreciable levels in early/less-severe CF (although some patients with early-stage disease report benefit), nor has DNase demonstrated clinical benefit for other obstructive pulmonary diseases. Clinical deterioration in lung function with DNase treatment is seen in 6-30% of pediatric patients. DNase can also exacerbate inflammation by promoting activity of neutrophil elastase, a proteolytic enzyme inhibited by the presence of the nucleic acids that DNase targets.

Despite aggressive use of DNase, response is low and CF disease typically progresses to bronchiectasis, respiratory failure, and death or transplant in childhood or early adulthood. While airway hydration/cough-inducing therapies such as mannitol or hypertonic saline inhalation have shown promise in clinical trials and some are now used in patient care, there is still a critical lack of effective mucus treatments, especially those that target pathologic mucus directly.

Unfortunately, results for the various thiol-containing small molecules that have been evaluated as mucus drugs have been disappointing. These agents include NAC and Nacystelyn (NAL; NAC+L-lysine) as well as reduced GSH and cysteamine. While largely safe, to date these small-molecule agents have not exhibited clear clinical benefits in either oral or inhaled forms.

Some of this poor efficacy may be the result of potency loss caused by autoxidation during inhalation delivery, as well as the potential for pulmonary enzymes to rapidly convert GSH to inactive forms, but the inherent low activity of non-enzyme thiol agents at acidic CF airway pH caused by their extremely basic thiol pKa's as described above may more likely be responsible.

As with mucus over-production, bicarbonate secretion defects and airway acidification are not restricted to CF but may also underlie other obstructive pulmonary diseases affecting large populations. Hence, truly effective and mechanistic mucus-modulating treatments will likely bring broad medical benefit. Improving thiol agents by combining disulfide-targeting with the superior potency, stability and specificity of biologic drugs like thioredoxin is highly desirable.

Unfortunately, development of formulation strategies for thiol-based therapeutics that can safely stabilize the protein in reduced form has been limited. Previous efforts (PCT WO2006/090127) required laborious screening of a large number of excipients to find a combination of sugars and chemical stabilizers that could enable compositions both able to remain reduced during prolonged storage in solid form and when reconstituted in liquid solutions for delivery. The reducing-sugar based formulations resulting from this strategy turned out to be markedly pro-inflammatory and hence unsuitable for use in inhalation delivery. Native thioredoxin reconstituted in saline using this sucrose formulation resulted in high levels of neutrophil influx and inflammatory cytokine release when delivered to rats by intratracheal administration (Rancourt, R. C., et al., 2007, Reduced thioredoxin increases proinflammatory cytokines and neutrophil influx in rat airways: modulation by airway mucus. Free Radic Biol Med 42, 1441-53). There thus remains a need for safe and effective formulation approaches for thiol-based protein therapeutics.

### BRIEF DESCRIPTION OF THE DRAWINGS

FIG. 1: Mechanism of mutation of Cysteine 35 to Serine (C35S TRX)

FIG. 2. pH dependence of thiol agent reducing activity vs. thioredoxin

FIG. 3. Stability of lyophilized solid forms and solutions of monothiol C35S thioredoxin

FIG. 4. UV spectra of main thioredoxin fraction (top) and red fraction with absorbance >400 nm (bottom) isolated by hydrophobic interaction chromatography

FIG. 5. Reduction in A. Elastic modulus (G′), B. Viscous modulus (G″) and C. Mucin Molecular Weight (GPC-MALLS) of 4% solids dry weight mucus reduced with DTT (1 mM) and ORP100S (0.01, 0.1, and 1.0 mM concentrations) for 1 hr at 37° C.

FIG. 6. μOCT analysis of ORP100S (“Theradux”) in primary HBE from CF patient donors

FIG. 7. μOCT analysis of ORP100S (“Theradux”) in CF patient sputum

FIG. 8. Effect of thioredoxin and C35S thioredoxin on levels of IL-6 or TNFalpha induced after 24 hr in the basolateral ALI media of primary HBE cultures from nasal epithelia of non-CF and CF donors

FIG. 9. Nebulized aerosol delivery of ORP-100 and ORP100S in rats and attenuation of formulation-induced neutrophil influx in vivo

### SUMMARY OF THE INVENTION

One aspect of the invention is a method to decrease viscoelasticity of mucus or sputum in a patient that has excessively viscous or cohesive mucus or sputum. The method includes contacting the mucus or sputum of the patient with a composition comprising a protein or peptide comprising a thioredoxin monocysteinic active site in a reduced state, where the protein or peptide does not contain any cysteine residues except for a single Cys at the N-terminal position of the thioredoxin monocysteinic active site.

In another aspect of the invention, a pharmaceutical composition is provided where the composition comprises a protein or peptide having a thioredoxin monocysteinic active site in a reduced state, wherein the protein or peptide does not contain any cysteine residues except for a single Cys at the N-terminal position of the thioredoxin monocysteinic active site and a pharmaceutically acceptable excipient.

A further aspect of the invention is a composition comprising a protein or peptide having a thioredoxin active site in a reduced state and an aqueous solvent having a vapor pressure of at least about 3 mmHg.

In yet another aspect of the invention, a pharmaceutical composition consisting essentially of a protein or peptide comprising a thioredoxin active site in a reduced state, water, and sodium chloride is provided.

Another aspect of the invention is a method of preparing a dried composition that includes providing an aqueous composition comprising a protein or peptide comprising a thioredoxin active site in a reduced state, and an aqueous solvent having a vapor pressure of at least about 3 mmHg. The method further includes volatilizing the aqueous solvent to produce a dried composition comprising the protein or peptide.

A still further aspect of the invention is a composition that consists essentially of or consists of a protein or peptide comprising a thioredoxin active site in a reduced state and normal saline.

Another aspect of the invention is a composition consisting essentially of a protein or peptide comprising a thioredoxin active site in a reduced state, where the composition is a dry powder.

Another method of the invention is a method to treat inflammation in a subject that includes administering to the subject a pharmaceutical composition comprising a protein or peptide comprising a thioredoxin monocysteinic active site in a reduced state, where the subject has or is at risk of developing inflammation.

A still further aspect of the invention is a method to treat bacterial infection in a subject by administering a pharmaceutical composition comprising a protein or peptide that has a thioredoxin monocysteinic active site in a reduced state, and where the subject has or is at risk of developing bacterial infection.

A further aspect of the invention is a composition comprising a thioredoxin monocysteinic active site operable to activate one or more endogenous antimicrobial peptides, wherein the activation results in a therapeutically effective reagent to treat or prevent infectious diseases.

A further method of the invention is a method to modulate the microbiome composition of a subject by topically administering to a mucosal surface of the subject a composition comprising a protein or peptide having a thioredoxin monocysteinic active site in a reduced state.

A still further method is for determining the disulfide bond reducing activity of a protein or peptide containing a monocysteinic thioredoxin active site, by selecting a protein or peptide containing a monocysteinic thioredoxin active site that does not contain any cysteine residue except for the single Cys in the thioredoxin monocysteinic active site; and measuring the overall cysteine thiol reduction state of the protein or peptide.

Yet another aspect of the invention is a method of treating a viral respiratory disease in a subject having or at risk of developing a viral respiratory disease by administering a composition comprising a protein or peptide comprising a thioredoxin monocysteinic active site in a reduced state to the subject.

The invention further includes a method of reducing lung inflammation associated with a viral infection in a subject in need thereof by administering to a subject in need thereof a pharmaceutical composition comprising a protein or peptide comprising a thioredoxin monocysteinic active site in a reduced state.

Another aspect of the invention is a composition comprising a protein or peptide having a thioredoxin active site, wherein the composition does not include a thioredoxin protein fraction having UV absorbance greater than about 400 nm wavelength.

A further aspect of the invention is a method to produce a composition comprising a protein or peptide comprising a thioredoxin active site, by providing a lysate comprising a protein or peptide comprising a thioredoxin active site; concentrating the protein or peptide in the lysate; and removing a thioredoxin peptide or protein fraction having absorbance greater than about 400 nm to produce the composition.

In various embodiments of the invention, the thioredoxin active site is a thioredoxin monocysteinic active site that comprises an amino acid sequence selected from the group consisting of C-X-X-S(SEQ ID NO: 24), C-X-X-X (SEQ ID NO: 17), X-C-X-X-X-X (SEQ ID NO: 19), X-C-G-P-X-X (SEQ ID NO: 21), W-C-G-P-X-K (SEQ ID NO: 23), X-C-X-X-S-X (SEQ ID NO: 25), X-C-G-P-S-X (SEQ ID NO: 26), and W-C-G-P-S-K (SEQ ID NO: 27), wherein X residues are any amino acid residue other than cysteine. In other embodiments, the protein or peptide comprises a sequence that is at least about 80% identical to SEQ ID NO:28 or SEQ ID NO:29, wherein the thioredoxin active site is a thioredoxin monocysteinic active site is at a position corresponding to positions 32-35 of SEQ ID NO:28 or SEQ ID NO:29. In still further embodiments, the protein or peptide comprises the sequence of SEQ ID NO:28 or SEQ ID NO:29. In other embodiments, the protein comprises human thioredoxin.

In some embodiments of the invention, the patient has a lung disease in which abnormal or excessive viscosity or cohesiveness of mucus or sputum is a symptom or cause of the disease. The patient can have a lung disease in which abnormal or excessive viscosity or cohesiveness of mucus or sputum is associated with a deficiency of biological reductant activity. In other embodiments, the patient has a disease selected from the group consisting of cystic fibrosis, chronic obstructive pulmonary disease, bronchiectasis, asthma, sinusitis, idiopathic pulmonary fibrosis, pulmonary hypertension, dry eye disease, and a digestive tract disease. In another embodiment, the patient has cystic fibrosis. In some embodiments, the patient is a human.

In embodiments of the method to decrease viscoelasticity of excessively viscous or cohesive mucus or sputum in a patient, the step of contacting the mucus or sputum of the patient with the composition is performed by introducing the composition to the patient by a route selected from the group consisting of nasal, intratracheal, bronchial, direct installation into the lung, inhaled, oral, and ocular. In other embodiments, the mucus or sputum to be contacted is in the respiratory tract of the patient. In other embodiments, after the step of contacting the mucus or sputum of the patient with the composition, the patient has at least about a 2.5% increase in forced expiratory volume (FEV) as compared to prior to the step of contacting.

In further embodiments, the protein or peptide containing a thioredoxin monocysteinic active site covalently binds to a cysteine residue in a mucus protein, such as where the mucus protein is a mucin, such as a respiratory mucus protein.

In embodiments of the invention, the composition can comprise a pharmaceutically acceptable carrier, and in such pharmaceutical compositions, the protein or peptide can comprise the thioredoxin monocysteinic active site sequence of SEQ ID NO: 1. Pharmaceutical compositions of the invention can be formulated for administration to a patient by a route selected from oral, rectal, nasal, inhaled, intratracheal, bronchial, direct instillation, topical, and ocular.

In embodiments of the invention having an aqueous solvent with a vapor pressure of at least about 3 mmHg, the aqueous solvent can be selected from ammonium acetate, ammonium bicarbonate, ammonium formate, triethylammonium acetate, and triethylammonium bicarbonate. The aqueous solvent can be ammonium acetate. In other embodiments, the aqueous solvent can be at a concentration of between about 1 mM and about 50 mM and/or the aqueous solvent can have a pH of between about 4 and about 7.

In some embodiments, compositions of the invention do not comprise a saccharide or saccharide derivative. In other embodiments, the aqueous composition does not contain any compound, other than the protein or peptide, having a vapor pressure of less than about 3 mmHg.

In other embodiments of the invention, the protein or peptide does not contain any cysteine residues except for one or two Cys in the thioredoxin active site. In still other embodiments, the thioredoxin active site can comprise an amino acid sequence selected from C-X-X-C(SEQ ID NO: 16), X-C-X-X-C-X (SEQ ID NO: 20), X-C-G-P-C-X (SEQ ID NO: 22), W-C-G-P-C-K (SEQ ID NO: 3), wherein X residues are any amino acid residue other than cysteine. In further embodiments, the thioredoxin active site is a monocysteinic thioredoxin active site, and the protein or peptide may not contain any cysteine residue except for a single Cys at the N-terminus of the thioredoxin monocysteinic active site.

In embodiments of the invention comprising sodium chloride, the sodium chloride can be present at about 9 grams of sodium chloride per 1 liter of water.

In embodiments comprising the step of volatilizing an aqueous solvent, the step of volatilizing can comprise subjecting the composition to a condition selected from the group consisting of reduced pressure, elevated temperature and combinations thereof. In such embodiments, the step of volatilizing can be done under a non-oxidizing atmosphere, such as a nitrogen atmosphere. In other embodiments, the step of volatilizing can include lyophilization.

In embodiments of the invention involving forming a dried pharmaceutical composition, the methods can include solubilizing the dried composition in a diluent, such as a saline solution having a pH between about 4 and about 7. Such solubilized pharmaceutical compositions can be at least 80% stable in the reduced form for at least about 1 day at a temperature of about 25° C., or at least 80% stable in the reduced form for at least about 1 week at a temperature of about 25° C. In other such embodiments, the thioredoxin can comprise a monocysteinic thioredoxin active site, or more particularly, a thioredoxin monocysteinic active site in a reduced state, wherein the protein or peptide does not contain any cysteine residue except for a single cysteine residue at the N-terminus of the thioredoxin monocysteinic active site.

In methods of the invention for treating inflammation, the administration of the protein or peptide can inhibit release of pro-inflammatory cytokines, such as pro-inflammatory cytokines are selected from IL-8, IL-1β, IL-6, and TNFα.

In methods of the invention for treating bacterial infection, the composition can comprise a crude or purified extract of microbial cells expressing the protein or peptide.

In embodiments of the invention comprising a composition to activate one or more endogenous antimicrobial peptides, the antimicrobial peptide can be a defensin.

In embodiments for modulating the microbiome composition of a subject, the mucosal surface can be a pulmonary surface, a nasopharyngeal surface, or a gastrointestinal surface.

In embodiments for determining the disulfide bond reducing activity of a protein or peptide, the cysteine thiol reduction state can be measured using a method selected from a chromogenic assay, a fluorometric assay, and a turbidometric assay. For example, chromogenic assay can be a DTNB assay.

In embodiments related to treating viral respiratory disease, the disease can be selected from Acute Respiratory Distress Syndrome (ARDS), Severe Acute Respiratory Distress Syndrome (SARS), Middle East Respiratory Syndrome (MERS), SARS-Coronavirus-2 (SARS-CoV-19 or COVID-19), influenza, viral infection associated with asthma, pneumonia, bronchitis, tuberculosis, reactive airway disease syndrome, and interstitial lung disease. In such embodiments, the viral respiratory disease can be caused by a virus selected from a coronavirus, an influenza virus, respiratory syncytial virus (RSV), a parainfluenza virus, and a respiratory adenovirus.

In various embodiments of the invention, the composition can be administered in a nebulized form or an aerosolized form, or in the form of a dry powder for inhalation.

In compositions of the invention having a protein or peptide with a thioredoxin active site, and not including a thioredoxin protein fraction having UV absorbance greater than about 400 nm wavelength, such compositions can further include an aqueous solvent having a vapor pressure of at least about 3 mmHg. Or, the compositions, can consist essentially of the protein or peptide comprising a thioredoxin active site in a reduced state, water and sodium chloride. Further, such compositions can be dried to a water content of less than about 3.0 wt. %.

In methods of the invention to produce a composition having a protein or peptide with a thioredoxin active site, removing a thioredoxin peptide or protein fraction having absorbance at greater than about 400 nm to produce the composition, the composition can be further characterized as having a peptide or protein fraction having absorbance at UV light wavelengths of less than about 300 nm. In such embodiments, the step of removing can include hydrophobic interaction chromatography and/or drying the composition to a water content of less than about 3.0 wt. %.

### DETAILED DESCRIPTION OF THE INVENTION

The present invention generally relates to the use of a thioredoxin protein or peptide containing a thioredoxin active site in a reduced state to treat mucosal diseases characterized by symptoms including one or more of abnormal mucus, inflammation, infection, or hypertension. More specifically, the present inventor has discovered that proteins or peptides with a thioredoxin active site, including thioredoxin proteins or peptides comprising a monocysteinic active site, decrease inflammation or the viscoelasticity and/or cohesiveness of abnormal sputum or mucus and thereby are effective agents for normalizing sputum or mucus.

Accordingly, proteins or peptides containing a thioredoxin active site in reduced state as stated above, or nucleic acid molecules encoding such proteins, can be used alone or in a composition to treat a variety of conditions or diseases associated with undesirable mucus or tenacious and viscous sputum as well as for treating inflammation, hypertension and/or infection. For example, respiratory diseases such as cystic fibrosis, chronic obstructive pulmonary disease, bronchiectasis, sinusitis, idiopathic pulmonary fibrosis, pulmonary hypertension, and asthma including status asthmaticus are particularly amenable to treatment using the product and process of the invention. Also, digestive tract diseases associated with thickened or adherent mucus such as coccidiosis are also particularly amenable to treatment using the product and process of the invention. Analogous diseases of other mucosal surfaces such as dry eye disease that are characterized by abnormally thickened mucus secretions, inflammation and/or infection are also amenable to treatment, as are ocular diseases involving oxidative stress and inflammation including macular degeneration, diabetic retinopathy, glaucoma, and cataract.

Therefore, the present invention relates to the use of proteins or peptides containing an active site of thioredoxin in a reduced state, including wherein the thioredoxin protein or peptide comprises a monocysteinic active site, for decreasing the viscoelasticity of mucus or sputum, particularly mucus or sputum that is abnormally or excessively viscous and/or cohesive. The proteins can be administered to a patient that is suffering from or affected by such abnormal or excessive mucus or sputum in a manner and amount effective to decrease the viscoelasticity of the mucus or sputum and preferably, to provide a therapeutic benefit to the patient. In addition, the proteins can be administered to a patient that is suffering from inflammation or infection, including patients having or at risk of runaway inflammatory responses, such as cytokine release syndrome (CRS), and associated acute lung injury such as acute respiratory distress syndrome (ARDS).

### Proteins and Peptides With a Thioredoxin Active Site

Thioredoxin-1 (Trx) is a small (12 kDa), naturally occurring redox protein for which protein disulfides are a preferred substrate. Trx is an essential human protein that plays a significant biological role in regulating protein and enzyme activity via potent and specific disulfide bond reduction.

Trx has a redox-active dithiol in its highly conserved Cys-Gly-Pro-Cys (SEQ ID NO:1) active site, which is reduced from the oxidized form by the flavoenzyme thioredoxin reductase (TrxR) and the cofactor NADPH. Together, these three components form the thioredoxin system whose reducing ability is many times more potent than small-molecule reducing agents.

By virtue of its role in reversible disulfide bond regulation, mammalian Trx is involved in numerous intracellular and extracellular redox signaling activities, including serving as a cofactor for methionine sulfoxide reductase, modifying DNA binding activities of receptors and transcription factors, and participating in protein folding. Furthermore, Trx can scavenge free radicals and is able to protect cells against oxidative stress, and secreted Trx is required at mucosal surfaces for activation (by disulfide bond reduction) of the important secreted antimicrobial human β-defensin-1, hBD-1.

The thioredoxin proteins or peptides as disclosed herein, have advantages over other reducing agents for use in the treatment of conditions such as cystic fibrosis. For example, unlike other reducing agents such as N-acetylcysteine (NAC), Nacystelyn (NAL), dithiothreitol (DTT), or reduced glutathione (GSH), the mutant thioredoxin disclosed herein is less susceptible to inactivation by enzymatic or auto-oxidative mechanisms, including reactions to produce superoxide, hydrogen peroxide, hydroxyl radical and other toxic oxygen metabolites. Furthermore, native or wildtype thioredoxin is a naturally-occurring compound which is normally secreted extracellularly onto the airway surface, and therefore, introduction of thioredoxin into the airway should be non-irritating and unlikely to induce an inappropriate immune response. Thioredoxin is also not glycosylated, and as such, it is more easily manufactured, and administration of the protein in natural or recombinant form should not induce an innate immune response. Perhaps even more significantly, reduced thioredoxin, in contrast to other reducing agents, more rapidly and potently restores the treated mucus or sputum to a normal viscosity level, and this normalization lasts for a longer duration. NAC, NAL, DTT, and GSH, for example, become “spent” or oxidized over time and at this stage, normalized sputum or mucus can revert back to an abnormal viscosity state. In contrast, the decrease in viscosity or viscoelasticity produced by thioredoxin appears to endure longer, most likely due to its cyclic re-reduction by its reducing system. Further, by remaining covalently bound to mucin Cys residues the monocysteinic active-site thioredoxin disclosed herein creates an even more potent and longer-duration reduction in viscosity compared to native thioredoxin. Finally, thioredoxin is both more potent and more specific for disulfide bond-reduction than other reducing agents and therefore, it can be used at significantly lower doses than other agents to achieve a beneficial effect.

As discussed above, thioredoxin (Trx) is a protein disulfide reductase that catalyzes numerous thiol-dependent cellular reductive processes. Native thioredoxin contains two redox-active cysteines that are highly conserved across species. In their oxidized form, these cysteines form a disulfide bridge that protrudes from the three dimensional structure of the protein. Protein disulfides are a preferred substrate for Trx-mediated reducing action. Modification of one of the two Trx active site cysteines to a residue other than cysteine produces a monocysteinic active site.

The present invention generally relates to the use of a thioredoxin protein or peptide containing a thioredoxin active site in a reduced state. Reference to “thioredoxin active site” includes either thioredoxin monocysteinic (i.e. monothiol) active site comprising the amino acid sequence C-X-X-X (SEQ ID NO:17) or native (or wild-type) thioredoxin dithiol active sites which contain two redox-active cysteines (an N-terminal cysteine and a C-terminal cysteine) comprising the amino acid sequence C-X-X-C having SEQ ID NO:16. As used herein, amino acid residues denoted “C” are cysteine residues and amino acid residues denoted “X” can be any amino acid residue other than a cysteine residue, and in particular, any of the remaining standard 20 amino acid residues or synthetic, unnatural or modified amino acids. The identity of X residues is independent of other X residues. That is, the identity of any X residue can be the same or different than other X residues.

A thioredoxin active site of the present invention can comprise the amino acid sequence C-G-P-X (SEQ ID NO:18), wherein the native or wild-type sequence comprises the amino acid sequence C-G-P-C(SEQ ID NO:1). A thioredoxin active site can further comprise the amino acid sequence X-C-X-X-X-X (SEQ ID NO:19), wherein the native or wild-type sequence comprises the amino acid sequence X-C-X-X-C-X (SEQ ID NO:20). In addition, a thioredoxin active site of the present invention comprises the amino acid sequence X-C-G-P-X-X (SEQ ID NO:21), wherein such amino acid residue denoted “G” is a glycine residue, and wherein such amino acid residue denoted “P” is a proline residue, wherein the native or wild-type sequence comprises the amino acid sequence X-C-G-P-C-X (SEQ ID NO:22). Another thioredoxin active site of the present invention comprises the amino acid sequence W-C-G-P-X-K (SEQ ID NO:23), wherein such amino acid residue denoted “W” is a tryptophan residue, and wherein such amino acid residue denoted “K” is a lysine residue and wherein the native sequence comprises the amino acid sequence W-C-G-P-C-K (SEQ ID NO:3). A thioredoxin active site can comprise the amino acid sequence C-X-X-S(SEQ ID NO:24). Such a thioredoxin active site of the present invention preferably comprises the amino acid sequence C-G-P-S(SEQ ID NO:1). A thioredoxin active site can further comprise the amino acid sequence X-C-X-X-S-X (SEQ ID NO:25), X-C-G-P-S-X (SEQ ID NO: 26) or W-C-G-P-S-K (SEQ ID NO:27), wherein amino acid residues denoted “X” can be any amino acid residue other than a cysteine residue. A monocysteinic thioredoxin active site can vary from a corresponding native sequence by substituting the C terminal cysteine of the native active site, as described above. In addition, a thioredoxin active site can vary by a deletion of the C-terminal cysteine of the native active site.

### Further Variants of Thioredoxin

A thioredoxin protein or peptide containing a thioredoxin active site can further comprise one or more cysteine deletions, substitutions or combinations thereof outside of the thioredoxin active site at non-active site cysteine residues. In one aspect, the one or more cysteines outside of the thioredoxin active site are substituted with any amino acid residue other than a cysteine residue. In one aspect, the one or more cysteines outside of the thioredoxin active site are substituted with any amino acid residue other than a cysteine or alanine residue. In one aspect, the one or more cysteines outside of the thioredoxin active site are substituted with a serine residue. In a further aspect, all of the non-active cysteines outside of the thioredoxin active site in the thioredoxin protein or peptide are deleted, substituted with a serine residue, or combinations thereof. In still a further aspect, all of the non-active cysteines outside of the thioredoxin active site in the thioredoxin protein or peptide are deleted and/or substituted with a serine residue or combinations thereof and the C-terminal cysteine in the thioredoxin active site is also substituted with a serine residue.

### Types of Thioredoxin

In one aspect of the invention, the thioredoxin protein containing a thioredoxin active site is a full-length thioredoxin protein or any fragment thereof containing a thioredoxin active site as described structurally and functionally above. Preferred thioredoxin proteins having active sites include prokaryotic thioredoxin, yeast thioredoxin, plant thioredoxin, and animal thioredoxin, with mammalian and human thioredoxin being further embodiments of animal thioredoxins. The nucleic acid and amino acid sequences of thioredoxin proteins from a variety of organisms are well known in the art and are intended to be encompassed by the present invention. For example, SEQ ID NOs:4-15 represent the amino acid sequences for thioredoxin from Pseudomonas syringae (SEQ ID NO:4), Porphyromonas gingivalis (SEQ ID NO:5), Listeria monocytogenes (SEQ ID NO:6), Saccharomyces cerevisiae (SEQ ID NO:7), Gallus (SEQ ID NO:8), Mus musculus (SEQ ID NO:9), Rattus norvegicus (SEQ ID NO:10), Bos taurus (SEQ ID NO:11), Homo sapiens (SEQ ID NO:12), Arabidopsis thaliana (SEQ ID NO:13), Zea mays (SEQ ID NO:14), and Oryza sativa (SEQ ID NO:15). Referring to each of these sequences, the C-X-X-C motif having SEQ ID NO:16 can be found as follows: SEQ ID NO:4 (positions 34-37), SEQ ID NO:5 (positions 29-32), SEQ ID NO:6 (positions 28-31), SEQ ID NO:7 (positions 30-33), SEQ ID NO:8 (positions 32-35), SEQ ID NO:9 (positions 32-35), SEQ ID NO:10 (positions 32-35), SEQ ID NO:11 (positions 32-35), SEQ ID NO:12 (positions 32-35), SEQ ID NO:13 (positions 60-63), SEQ ID NO:14 (positions 89-92) and SEQ ID NO:15 (positions 95-98).

### Modifications Outside of the Active Site

Referring to SEQ ID NO:12, non-active cysteine residues outside of the thioredoxin active site that can be deleted and/or substituted can be found at positions 62, 69 and 73 of the human thioredoxin-1 sequence. Again referring to SEQ ID NO:12, the active site Cys are found at positions 32 and 35, with the cysteine at position 32 referred to as the N-terminal cysteine and the cysteine at position 35 referred to as the C-terminal cysteine. In one aspect, the cysteines at positions 62, 69 and 73 of SEQ ID NO:12, or cysteines at corresponding positions in other thioredoxins, are deleted, substituted or a combination thereof with any amino acid residue other than a cysteine residue. In still another aspect, the cysteines at positions 62, 69 and 73 of SEQ ID NO:12, or cysteines at corresponding positions in other thioredoxins, are substituted with any amino acid residue other than a cysteine residue or an alanine residue. In one further aspect, the cysteines at positions 62, 69 and 73 of SEQ ID NO:12, or cysteines at corresponding positions in other thioredoxins, are substituted with serine. In yet another further aspect, the cysteines at positions 35, 62, 69 and 73 of SEQ ID NO:12, or cysteines at corresponding positions in other thioredoxins, are substituted with serine. In one aspect, the cysteines at positions 62, 69 and 73 of SEQ ID NO:12, or cysteines at corresponding positions in other thioredoxins, are deleted and the cysteine at position 35 of SEQ ID NO:12, or the cysteine at a corresponding position in other thioredoxins, is substituted with any amino acid residue other than a cysteine residue and preferably is substituted with a serine residue. In still another aspect, the cysteines at positions 62, 69 and 73 of SEQ ID NO:12, or cysteines at corresponding positions in other thioredoxins, are deleted and/or substituted with any amino acid residue other than a cysteine residue and/or a combination thereof and the cysteine at position 35 of SEQ ID NO:12, or the cysteine at a corresponding position in other thioredoxins, is substituted with any amino acid residue other than a cysteine residue and preferably is substituted with a serine residue.

### Single Cysteine Thioredoxin

In a particular embodiment of the invention, the thioredoxin protein is a protein or peptide, comprising a thioredoxin monocysteinic active site in a reduced state, wherein the protein or peptide does not contain any cysteine residue except for a single cysteine residue in the thioredoxin monocysteinic active-site, such as shown as SEQ ID NO:28 and SEQ ID NO:29 in which the thioredoxin protein is a fully monocysteinic variation of SEQ ID NO:12 in which the sole cysteine is at position 32 (or more generally at the N terminal position of the thioredoxin active site). This embodiment of the invention also includes variants of SEQ ID NO:28 and SEQ ID NO:29 having amino acids that are substituted and/or deleted, while still being fully monocysteinic and having a sole cysteine at position 32 (or more generally at the N terminal position of the thioredoxin active site). Such variants can have 1, 2, 3, 4, 5, 6, 7, 8, 9, 10, 11, 12, 13, 14, 15, 16, 17, 18, 19, or 20 such substitutions and/or deletions to the sequence of SEQ ID NO:28 or SEQ ID NO:29. In alternative embodiments, the variants can be characterized by having at least about 80% identity to SEQ ID NO:28 or SEQ ID NO:29, at least about 85% identity to SEQ ID NO:28 or SEQ ID NO:29, at least about 90% identity to SEQ ID NO:28 or SEQ ID NO:29, at least about 95% identity to SEQ ID NO:28 or SEQ ID NO:29, at least about 99% identity to SEQ ID NO:28 or SEQ ID NO:29, or at least any whole number percent identity between 80% and 99%.

The three-dimensional structure of several thioredoxin proteins has been resolved, including human and bacterial thioredoxins. Therefore, the structure and active site of thioredoxins from multiple organisms is well known in the art and one of skill in the art would be able to readily identify and produce fragments or homologues of full-length thioredoxins, including thioredoxins having monocysteinic active sites in combination with deletions, substitutions or combinations thereof of the non-active cysteine residues outside of the thioredoxin active site that can be used in the present invention. Examples of thioredoxin proteins include ORP-100 which is a thioredoxin protein having a monothiol active site in which the second active site cysteine at position 35 (i.e. the C-terminal cysteine) has been replaced with a serine residue. ORP100S is a thioredoxin protein having a monothiol active site in which the second active site cysteine at position 35 (i.e. C-terminal cysteine) has been substituted with a serine and wherein all the non-active cysteine residues outside of the thioredoxin active site (found at positions 62, 69 and 73) have also been substituted with serine residues (SEQ ID NO:29).

### Reduced Cysteines

The phrase “in a reduced state” specifically describes the state of the cysteine residues in the active site of a protein or peptide of the present invention. In a reduced state, adjacent cysteine residues form a dithiol (i.e. two free sulfhydryl groups, —SH). In contrast, in oxidized form, such cysteine residues form an intramolecular disulfide bridge; such a molecule can be referred to as cystine. In a reduced state, a thioredoxin active site is capable of participating in redox reactions through the reversible oxidation of its active site thiol to a disulfide, and catalyzes thiol-disulfide exchange reactions that result in covalent linkage to one of the target disulfide cysteines. For proteins or peptides of the present invention containing a thioredoxin monothiol active site and further comprising deletion, substitution or combinations thereof of one or more cysteine residues outside of the thioredoxin active site with any amino acid residue other than a cysteine, the N-terminal cysteine in the active site is in a reduced state as a monothiol and is therefore able to form a stable mixed-disulfide with a cysteine on the target protein.

### Protein or Peptide Sizes

As used herein, a protein or peptide of the present invention containing a thioredoxin active site can be a thioredoxin active site per se or a thioredoxin active site joined to other amino acids by glycosidic linkages. Thus, the minimal size of a protein or peptide of the present invention is from about 4 to about 6 amino acids in length, with preferred sizes depending on whether a full-length, fusion, multivalent, or merely functional portions of such a protein is desired. Preferably, the length of a protein or peptide of the present invention extends from about 4 to about 100 amino acid residues or more, with peptides of any interim length, in whole integers (i.e., 4, 5, 6, 7 . . . 99, 100, 101 . . . ), being specifically envisioned. It may also be a short thioredoxin mimetic peptide blocked at the N and C termini as described by Bachnoff et al., Free Radical Biol Med 50:1355-67, 2011.

### Homologues

In a further preferred embodiment, a protein of the present invention can be a full-length protein or any homologue of such a protein. As used herein, the term “homologue” is used to refer to a protein or peptide which differs from a naturally occurring protein or peptide (i.e., the “prototype” or “wildtype” protein) by modifications to the naturally-occurring protein or peptide, but which maintains the basic protein and side chain structure of the naturally-occurring form, and/or which maintains a basic three-dimensional structure of at least a biologically active portion (e.g., the thioredoxin active site) of the native protein. Such changes include, but are not limited to: changes in one or a few amino acid side chains; changes in one or a few amino acids, including deletions (e.g., a truncated version of the protein or peptide (fragment)), insertions and/or substitutions; changes in stereochemistry of one or a few atoms; and/or minor derivatizations, including but not limited to: methylation, glycosylation, phosphorylation, acetylation, myristoylation, prenylation, palmitoylation, amidation and/or addition of glycosylphosphatidyl inositol. According to the present invention, any protein or peptide useful in the present invention, including homologues of natural thioredoxin proteins, have a thioredoxin monthiol active site such that, in a reduced state, the protein or peptide is capable of participating in redox reactions through the oxidation of its active site thiol to a disulfide and/or of decreasing the viscoelasity or cohesiveness of mucus or sputum or increasing the liquefaction of mucus or sputum.

As used herein, a protein or peptide containing a thioredoxin active site and further comprising deletion and/or substitution and/or combinations thereof of one or more cysteine residues outside of the thioredoxin active site with any amino acid residue other than a cysteine, can have characteristics similar to thioredoxin, and preferably, is a thioredoxin selected from the group of prokaryotic thioredoxin, fungal thioredoxin (including yeast), plant thioredoxin, animal thioredoxin, or mammalian thioredoxin. In a particularly preferred embodiment, the protein is human thioredoxin.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications in homologues, as compared to the wild-type protein, either agonize, antagonize, or do not substantially change, the basic biological activity of the homologue as compared to the naturally occurring protein. In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally-occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

### Preparation of Thioredoxin Compositions

Due to the structural stability and physical robustness characteristic of thioredoxins, the primary formulation development goal was maintenance of stored protein in the fully reduced, active form. As an initial approach protein reduced using DTT was exchanged into PBS, degassed with nitrogen to remove oxygen, and frozen in single-use aliquots at −80° C. to avoid successive freeze-thaw cycles. This strategy required that significant care be taken during storage and use and was not optimal since the protein rapidly oxidized in aqueous solution even when deep frozen. Other formulations were evaluated based on extensive work conducted at Syngenta Corp and Octoplus (a formulation development specialist) that utilized various complex combinations of saccharides and chemical excipients in an effort to stabilize the reduced state of native Trx in a dry storage formulation. Only one of these formulations (9% sucrose, 1.7 mM EDTA, pH 5.2) was found to confer suitable redox stability to thioredoxin following lyophilization, resulting in almost complete retention of starting activity even during accelerated storage at 40° C. for six months. However, this complex formulation raised concerns regarding the potential for inflammation when inhaled, and the high concentration of sucrose increased solution viscosity adversely and made isotonic reconstitution in suitable buffers challenging at protein concentrations required for drug delivery. Despite extensive experimentation no benign formulation was reported that could suitably maintain thioredoxin in the reduced state without oxidation, dimerization or multimerization.

The present inventors had the breakthrough realization that a formulation approach utilizing a volatile solvent, such as 20 mM ammonium acetate (pH 5.5) might allow thioredoxin proteins or peptides of the invention to be frozen in the reduced form and lyophilized, during which process the solvent would evaporate completely leaving only pure protein in the lyophilizate. This contrary approach was found surprisingly to confer beneficial redox stabilization properties to reduced thioredoxin even superior to the complex sucrose formulation of Syngenta, but without the proinflammatory effects of sucrose and EDTA. Moreover, by eliminating residual excipients in the lyophilized material the stable thioredoxin could be reconstituted into any desired buffer without concern for alteration of tonicity, enabling stable solution concentrations exceeding 5-10 mM.

A further embodiment of the invention is a method of preparing a composition that is useful for storage and transport of thioredoxin proteins and peptides of the invention. Such compositions are useful for preparing pharmaceutical compositions comprising thioredoxin proteins and peptides of the invention for administration to patients. In particular, the thioredoxin proteins and peptides of the invention can be stably stored and transported in reduced state in a minimal formulation without complex stabilizers or other formulation requirements. Consequently, reconstituted thioredoxin proteins and peptides of the invention prepared from such compositions can be in a minimal naked formulation without complex formulation requirements or the need for any excipients. The inventors have found the surprising and unexpected result that this method allows retention of protein and redox stability that is as good as or better than that obtained using complex, non-volatile excipients that required significant experimentation to derive, including for example, the sucrose-EDTA formulation as described in WO2006/090127.

The method includes providing a composition comprising a protein or peptide comprising a thioredoxin active site in a reduced state and an aqueous solvent having a vapor pressure of at least about 3 mmHg. The method then includes volatilizing the aqueous solvent to produce a dried composition comprising the protein or peptide. Such compositions and resulting pharmaceutical compositions are substantially free of contaminants, such as diluents, solvents, other solutions or liquids, buffers, salts, surfactants, and other chemicals. Such compositions can consist of the protein or peptide or can consist essentially of the protein or peptide. The basic and novel properties of such compositions include one or more of the characteristics that the thioredoxin active site of the protein or peptide is in a reduced state, lacks the significant ability to undergo spontaneous oxidation or lacks the significant ability to undergo spontaneous dimerization and/or that once reconstituted in isotonic saline, the protein or peptide is active in reduced form (i.e., can form stable disulfide bonds with targets). In the case of a monocysteinic active site thioredoxin it is further able to covalently bind a Cys residue of a target protein disulfide which attenuates the ability for the thioredoxin to be taken up intracellularly in an active form.

The term “solvent” as used herein refers to the liquid or solution into which a protein or peptide of the invention is suspended and/or dissolved prior to removing the solvent by volatilization, such as by lyophilization. The term “diluent” as used herein refers to the liquid or solution into which the protein or peptide of the invention is reconstituted after solvent removal by volatilization. Such reconstitution can result in the protein or peptide of the invention being suspended or dissolved in the diluent.

Preparation of a composition by this method to produce a protein or peptide of the invention can be accomplished by starting with a protein or peptide of the invention suspended or dissolved in a solvent. The solvent can have a vapor pressure suitable for lyophilization of the thioredoxin, for example, at least about 3 mmHg. The vapor pressure can also be at least about 1 mmHg, 2 mmHg, 3 mmHg, 4 mmHg, 5 mmHg, 6 mmHg, or 7 mmHg or at least about any decimal number between 1 mmHg and 7 mmHg. In other embodiments, the vapor pressure of the aqueous solvent can be in a range defined by any two values between 1 mmHg and 10 mmHg. Volatilization can then be performed according to suitable methods, such as lyophilization by standard protocols to produce a protein or peptide of the invention in reduced state that is free of solvent or other liquid. Such a resulting protein or peptide can be substantially pure (e.g., at least about 95, 96, 97, 98, 99, 99.5, 99.9%, or 100% pure).

In some embodiments the aqueous solvent can be selected from the group consisting of ammonium acetate, ammonium bicarbonate, ammonium formate, triethylammonium acetate, and triethylammonium bicarbonate. The aqueous solvent can be at a concentration of between about 1 mM and about 50 mM, or any whole number range between 1 mM and about 50 mM, and/or have a pH of between about 4 and about 7, or any decimal number range between about 4 and about 7. The composition having protein or peptide comprising a thioredoxin active site in a reduced state and an aqueous solvent having a vapor pressure of at least about 3 mmHg does not contain a saccharide or saccharide derivative in some embodiments, and in some embodiments it does not contain any compound, other than the protein or peptide, having a vapor pressure of less than about 3 mmHg.

The mixture of solvent and protein or peptide of the invention that is provided according to this method can consist of substantially only a single solvent and the protein or peptide of the invention. The mixture can also consist of the protein or peptide of the invention and multiple solvents (i.e., a mixed solvent) that collectively meet the vapor pressure limitations of this method. The mixture can also contain more than one protein or peptide of the invention, for example, more than one of the variants of thioredoxin disclosed herein and/or other proteins or peptides.

Alternatively, the mixture of a protein or peptide of the invention and solvent can consist essentially of the protein or peptide of the invention and solvent. The basic and novel characteristic of such embodiments is that components of the mixture other than the protein or peptide of the invention can be volatilized while the protein or peptide of the invention remains in a reduced state. In this manner, the protein or peptide of the invention can be stably preserved in a reduced state and is lyophilized into a state that is stable for storage and easily reconstituted in a reduced state for pharmaceutical and/or non-pharmaceutical uses.

An important aspect of the lyophilization procedure is that it can produce stable protein or peptide of the invention in a reduced state. If the starting material for lyophilization is reduced thioredoxin, the process described herein can preserve the thioredoxin active site cysteine in a reduced state and yield substantially pure thioredoxin with the active site cysteine in a reduced state. Furthermore, the thioredoxin active site cysteine can be preserved in a reduced state, regardless of whether other cysteines are present in the thioredoxin.

The substantially pure protein or peptide of the invention with the active site cysteine in a reduced state is then suitable for storage and is more amenable to storage at various temperatures and for various durations than protein compositions prepared by other methods. For example, the protein or peptide of the invention prepared by this method can be stable (e.g., with the active site cysteine in a reduced state), such as with greater than about 50% activity, greater than about 60% activity, greater than about 70% activity, greater than about 80% activity, greater than about 90% activity, or greater than about 95% activity. Such activity levels can be achieved for at least about 3 hours, at least about 6 hours, at least about 12 hours, at least about 1 day, at least about 2 days, at least about 3 days, at least about 4 days, at least about 5 days, at least about 6 days, at least about 7 days, at least about 2 weeks, or at least about 1 month. Additionally, the resulting composition can be stored between −80° C. to 40° C. and is able to retain thioredoxin reducing activity.

Compositions produced by this method can be substantially salt-free, such as with less than about 0.01, 0.1, 0.5, 1, 2, 3, 4, 5, or 10 mM salt. In addition, the lyophilized compositions can be further characterized as comprising at least about 90%, 91%, 92%, 93%, 94%, 95%, 96%, 97%, 98% or 99% by weight protein or peptide of the invention.

Proteins or peptides of the present invention prepared as above can then be reconstituted for various uses, including pharmaceutical and non-pharmaceutical uses. Reconstitution can be accomplished by resuspending or dissolving the protein or peptide of the invention in a suitable diluent. For example, the protein or peptide of the invention can be reconstituted with sterile water, isotonic saline, hypertonic saline, phosphate-buffered saline (PBS), combinations thereof or other diluents suitable for reconstitution. In some embodiments, the diluent is suitable for administration to a patient, such as a human patient, but also including other animals, including non-human mammals, birds, fish, reptiles and amphibians.

Alternatively, the protein or peptide of the invention prepared as above can be used without reconstitution. For example, the protein or peptide of the invention can be administered as a powder or as a dry component in food or tablets. Other uses of the non-reconstituted thioredoxin are possible.

The methods of preparation described herein can be used for any of the thioredoxin variants, fragments, active sites, homologues, or other versions of thioredoxin described herein.

This method and the resulting formulation developed by the inventors utilizes volatile solvents resulting in pure dried protein following lyophilization. This process allows reconstitution in components such as isotonic buffered saline (PBS), which dramatically simplifies the formulation and delivery process. Remarkably, the new lyophilized protein has equivalent redox stability similar to a sucrose-based formulation while eliminating the potential inflammatory risk of inhaled sucrose/EDTA. This new formulation has allowed elimination of all excipients in the final lyophilized material, facilitating reconstitution in simple isotonic saline buffer for delivery by a device such as an electronic vibrating-mesh nebulizer. The resulting formulation is simple and has the compelling advantage of being a salt/excipient-free formulation vs. the potential safety and delivery/efficiency challenges of a sucrose-containing inhaled drug product. Preliminary stability data indicate that it may be equivalent or superior to the original sucrose formulation.

In a further embodiment, the invention includes a composition comprising a protein or peptide comprising a thioredoxin active site, wherein the composition does not include a thioredoxin protein fraction having ultraviolet (UV) absorbance at greater than about 400 nm and methods of making the same. It has been surprisingly found that after production of proteins of the present invention, purification of a protein fraction having thioredoxin activity involving removal of a protein fraction having absorbance at light wavelengths of greater than about 400 nm increases the stability of the peptide or protein in the composition whether in dried (lyophilized) form or compositions that have been reconstituted, for example, in saline buffer from a dried form. Such peptide or protein compositions have also been found to be able to be dried to lower water contents, such as below about 5.0 wt. %, 4.0 wt. %, 3.0 wt. %, or less than about any 0.1 wt. % increment between 5.0 wt. % and 1.0 wt. %.

In some embodiments, the remaining peptide or protein fraction in the composition has absorbance at light wavelengths of less than about 400 nm, less than about 390 nm, less than about 380 nm, less than about 370 nm, less than about 360 nm, less than about 350 nm, less than about 340 nm, less than about 330 nm, less than about 320 nm, less than about 310 nm, less than about 300 nm, less than about 290 nm, or less than about 280 nm.

Embodiments of the invention including a composition comprising a protein or peptide comprising a thioredoxin active site, wherein the composition does not include a fraction having absorbance at greater than about 400 nm can be in various formats as described elsewhere herein. For example, such compositions can be in a form suitable for lyophilization wherein the protein or peptide comprising a thioredoxin active site is in a reduced state and wherein the composition further comprises an aqueous solvent having a vapor pressure of at least about 3 mmHg. Alternatively, such compositions can be in a dried state having a low water content as described above. Further, such compositions can be reconstituted so they are suitable for administration such as in a composition that consists essentially of the protein or peptide comprising a thioredoxin active site in a reduced state, water and sodium chloride.

Other embodiments include methods of making a composition comprising a protein or peptide comprising a thioredoxin active site, wherein the composition does not include a fraction having absorbance at greater than about 400 nm. Such methods include providing a lysate comprising a protein or peptide comprising a thioredoxin active site; concentrating the protein or peptide in the composition; and removing a peptide or protein fraction having absorbance at greater than about 400 nm. For example, and without limitation, proteins of the present invention can be produced by recombinant production in a host cell. The cells can be lysed and clarified. The resulting clarified composition can be subjected to further purification such as ion exchange chromatography. It has been found that a fraction having absorbance at greater than about 400 nm is not separated from the remaining main protein fraction having thioredoxin activity and absorbance at wavelengths less than about 400 nm, such as at about 280 nm by an ion exchange chromatography step. However, subjecting this fraction to hydrophobic interaction chromatography results in separation of the main protein fraction from a fraction having absorbance at greater than about 400 nm. The resulting main protein fraction has the beneficial attributes described above of a lower water content when dried and having increased stability (as measured for example by free SH groups and percent monomers).

### Pharmaceutical Compositions

The present invention also relates to pharmaceutical compositions comprising a solution of a protein or peptide containing a thioredoxin active site in a reduced state. In one embodiment, the protein or peptide does not contain any cysteine residue except for a single cysteine residue in the thioredoxin monocysteinic active site. Such pharmaceutical compositions also include a pharmaceutically acceptable excipient. In various embodiments, the excipient can be selected from ammonium acetate buffer, formic acid, acetic acid with ammonium, acetic acid without ammonium and combinations thereof.

In some embodiments, the thioredoxin monocysteinic active site comprises an amino acid sequence selected from the group consisting of C-X-X-S(SEQ ID NO: 24), C-X-X-X (SEQ ID NO: 17), X-C-X-X-X-X (SEQ ID NO: 19), X-C-G-P-X-X (SEQ ID NO: 21), W-C-G-P-X-K (SEQ ID NO: 23), X-C-X-X-S-X (SEQ ID NO: 25), X-C-G-P-S-X (SEQ ID NO: 26), and W-C-G-P-S-K (SEQ ID NO: 27), wherein the X residues are any amino acid residue other than cysteine. In other embodiments, the protein or peptide comprises the thioredoxin monocysteinic active site sequence of SEQ ID NO:1. In a further embodiment, the protein or peptide comprises a sequence that is selected from SEQ ID NO:28 and a sequence having at least about 80% identity to SEQ ID NO:28, where the thioredoxin monocysteinic active site is at a position corresponding to positions 32-35 of SEQ ID NO:28. In a further embodiment, the protein or peptide comprises a sequence that is selected from SEQ ID NO:29 and a sequence having at least about 80% identity to SEQ ID NO:29, where the thioredoxin monocysteinic active site is at a position corresponding to positions 32-35 of SEQ ID NO:29.

Such pharmaceutical compositions can be formulated for administration to a patient by a route selected oral, rectal, nasal, intratracheal, bronchial, direct installation into the lung, inhaled, oral, topical, and ocular.

### Administration

Additionally, a composition, including a pharmaceutical composition of the present invention can be administered to a patient in a pharmaceutically acceptable carrier. As used herein, a pharmaceutically acceptable carrier refers to any substance suitable for delivering a therapeutic protein, nucleic acid or other compound useful in the method of the present invention to a suitable in vivo or ex vivo site. Preferred pharmaceutically acceptable carriers are capable of maintaining a protein, nucleic acid molecule or compound in a form that, upon arrival of the protein, nucleic acid molecule or compound at the desired site (e.g., the site where the mucus or sputum to be treated is secreted or drains), is capable of contacting the mucus or sputum (in the case of a protein or compound) or of entering the cell and being expressed by the cell and secreted (in the case of a nucleic acid molecule) so that the expressed protein in a reduced state can contact the mucus or sputum.

A suitable, or effective, amount of a thioredoxin protein or peptide containing a thioredoxin active site as disclosed herein to administer to a patient is an amount that is capable of: participating in redox reactions through the reversible oxidation of its active site thiol to a disulfide, catalyzing thiol-disulfide exchange reactions, and particularly, decreasing the viscoelasticity or cohesiveness of mucus or sputum and/or increasing the liquefaction of mucus or sputum in a patient, sufficient to provide a therapeutic benefit to the patient. Decreases in the viscoelasticity or cohesiveness or increases in the liquefaction of mucus or sputum can be measured, detected or determined as described previously herein or by any suitable method known to those of skill in the art. As discussed above, such measurements include determining and comparing the percentage of free thiols in a sample of mucus or sputum from the patient prior to after contact with a suitable or effective amount of a protein or peptide containing a thioredoxin monocysteinic active site, as well as determining and comparing the FEV level of the patient prior to after contact with a suitable or effective amount of a protein or peptide containing a thioredoxin monocysteinic active site in a reduced state.

Besides decreasing viscoelasticity of mucus or sputum, the thioredoxin protein or peptide having a thioredoxin active site in a reduced state as disclosed herein, also has therapeutic uses such as treatment of inflammation, hypertension, oxidative stress or infection wherein the thioredoxin protein or peptide is administered topically by inhalation, direct application, instillation, or by oral administration; or, alternatively, by infusion, injection, or other routes of administration suitable for systemic extracellular treatment.

Methods for determining the activity of a thioredoxin protein in a reduced state formulated in a pharmaceutically-acceptable solution comprise determining the redox state of cysteines in the thioredoxin protein by an assay such as a fluorometric assay and/or a colorimetric assay, such as a DTNB assay (uses 5,5′-dithiobis-(2-nitrobenzoic acid). In one aspect, the thioredoxin protein comprises a single cysteine amino acid. In one aspect, the redox state of the N-terminal cysteine in the thioredoxin active site is determined by a fluorometric assay and/or a colorimetric assay, such as a DTNB assay.

In one embodiment, a suitable, or effective, amount of a thioredoxin protein or peptide containing a thioredoxin active site as disclosed herein to be administered to a patient comprises between about 10 μmoles/kg, 15 μmoles/kg, 20 μmoles/kg, 25 μmoles/kg, 30 μmoles/kg, 35 μmoles/kg, 40 μmoles/kg, 45 μmoles/kg, 50 μmoles/kg, 55 μmoles/kg, 60 μmoles/kg, 65 μmoles/kg, 70 μmoles/kg, 75 μmoles/kg, 80 μmoles/kg, 85 μmoles/kg, 90 μmoles/kg, 95 μmoles/kg, 100 μmoles/kg, 105 μmoles/kg, 110 μmoles/kg, 115 μmoles/kg, 120 μmoles/kg, 125 μmoles/kg, 130 μmoles/kg, 135 μmoles/kg, 140 μmoles/kg, 145 μmoles/kg, 150 μmoles/kg, 175 μmoles/kg, 200 μmoles/kg, 225 μmoles/kg, 250 μmoles/kg, 275 μmoles/kg, 300 μmoles/kg, 325 μmoles/kg, 350 μmoles/kg, 375 μmoles/kg, 400 μmoles/kg, 425 μmoles/kg, 450 μmoles/kg, 475 μmoles/kg, 500 μmoles/kg, 525 μmoles/kg, 550 μmoles/kg, 575 μmoles/kg, 600 μmoles/kg, 625 μmoles/kg, 650 μmoles/kg, 675 μmoles/kg, 700 μmoles/kg, 725 μmoles/kg, 750 μmoles/kg, 775 μmoles/kg, 800 μmoles/kg, 825 μmoles/kg, 850 μmoles/kg, 875 μmoles/kg, 900 μmoles/kg, 925 μmoles/kg, 950 μmoles/kg, 975 μmoles/kg, 1000 μmoles/kg, 1100 μmoles/kg, 1200 μmoles/kg, 1300 μmoles/kg, 1400 μmoles/kg, 1500 μmoles/kg, 1600 μmoles/kg, 1700 μmoles/kg, 1800 μmoles/kg, 1900 μmoles/kg, 2000 μmoles/kg, 2100 μmoles/kg, 2200 μmoles/kg, 2300 μmoles/kg, 2400 μmoles/kg or about 2500 μmoles/kg body weight of a patient.

In another embodiment, if the route of delivery is aerosol delivery to the lung or a similar route, an amount of a thioredoxin protein or peptide containing a thioredoxin active site as disclosed herein to be administered to a patient comprises between about 0.25 mg per dosing unit (e.g., a dosing unit for a human is typically about 2-3 ml) to about 100 mg per dosing unit, such that an effective concentration of at least 100 uM is achieved at the target site. Preferably, an amount of a thioredoxin protein or peptide containing a thioredoxin active site as disclosed herein to be administered to a patient comprises about 0.25 mg, 0.50 mg, 1.0 mg, 5.0 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or about 100 mg per dosing unit. Depending on the device used for aerosol delivery, some aerosol delivery devices only allow for about 10% of the volume in the aerosol to actually be delivered to the lung. However, when the delivery device is a vibrating mesh nebulizer, about 90% of the volume in the aerosol can be delivered. Electronic vibrating-mesh nebulizers, are capable of delivering drugs far more rapidly and are smaller, more portable devices that are greatly preferred by CF patients (Geller, D. E., Pediatric Pulmonology, 43(S9):S5-S17, 2008). Vibrating-mesh nebulizers also are more efficient at delivering drugs with less residual dose vs. air-jet nebulizers. This is particularly significant for reducing treatment costs as smaller doses are required to achieve therapeutic benefit. Devices such as these also do not result in reduced biological activity of proteins (Kesser, K. C., et al. Resp Care, 54(6):754-768, 2009; Scherer, T., et al. J Pharm Sci, 100(1):98-109, 2011). Therefore, for other routes of administration when the volume of the composition that will be delivered to the site is greater, it will readily be seen that lower doses of the protein or peptide comprising a thioredoxin active site may be used.

The optimum amount of a protein of the present invention to be administered to an animal will vary depending on the route of administration. For instance, if the protein is administered by an inhaled (aerosol) route, the optimum amount to be administered may be different from the optimum amount to be administered by intratracheal microspray. It is within the ability of one skilled in the art to vary the amount depending on such route of administration. It is important to note that a suitable amount of a protein of the present invention is an amount that has the desired function without being toxic to an animal. Other routes of administration include but are not limited to oral administration, especially for the treatment of digestive mucus, or topical for the treatment of reproductive mucus.

In a one embodiment of the present invention, a composition, including a pharmaceutical composition, of the present invention that contains a thioredoxin protein comprising a thioredoxin active site as disclosed herein is further formulated for delivery with one or more agents that maintains the thioredoxin active site in a reduced state following initial reduction using reducing agents. Such reducing agents used in the present invention include, but are not limited to, dithithreitol (DTT), lipioc acid, NADH or NADPH-dependent thioredoxin reductase, ethylenediaminetetraacetic acid (EDTA), reduced glutathione, dithioglycolic acid, 2-mercaptoehtanol, Tris-(2-carboxyethyl)phoshene, N-acetyl cysteine, NADPH, NADH and other biological or chemical reductants. As described herein, preferable lyophilized storage formulations for reduced thioredoxin were surprisingly found to not require any formulation excipients, although specific delivery formulations for certain mucosal or epithelial targets may benefit.

### Therapeutic Aspects

There are several advantages and benefits of thioredoxin proteins or peptides disclosed herein versus the native or wildtype thioredoxin. The active site modification of substituting the C-terminal cysteine with any amino acid residue other than a cysteine along with deletion and/or substitution or a combination thereof of one or more cysteine residues outside of the thioredoxin active site with any amino acid residue other than a cysteine, is designed to minimize potential side effects of thioredoxin associated with intracellular signaling or systemic exposure such as those described by Rancourt et al. (Free Radical Biol & Med 42:1441-43, 2007). These modifications prevent nucleophilic attack on the mixed disulfide formed between thioredoxin and a target protein disulfide that is catalyzed by the N-terminal thioredoxin active site cysteine (for example located at position 32 in human thioredoxin, SEQ ID NO:12).

Surprisingly, the present inventor has determined that such a thioredoxin has greater potency than wildtype thioredoxin decreasing (trending toward liquefying) and normalizing the viscoelasticity of diseased human mucus. The present inventors have found that not only does the thioredoxin containing a monothiol active site along with deletion and/or substitution and/or combination thereof of one or more cysteine residues outside of the thioredoxin active site with any amino acid residue other than a cysteine, not show impaired activity compared to wild-type thioredoxin, it exhibits greater stability and quantitative ability to reduce human CF mucus viscosity in a rheological assay especially as compared to thioredoxin that still retains the three non-active-site Cys residues at positions 62, 69 and 73.

Mucus obstruction of the airways can cause significant morbidity and mortality in patients with CF. The present inventor has demonstrated that the viscoelastic properties facilitating the persistence of these secretions within airways are markedly diminished by the thioredoxin proteins or peptides disclosed herein, and that dosing even as high as 40 mg/kg in rats does not cause adverse effects.

Accordingly, one embodiment of the present invention relates to a method to normalize and decrease the viscoelasticity of mucus or sputum in a patient that has excessively viscous or cohesive mucus or sputum. The method includes the step of contacting the mucus or sputum of the patient with a composition comprising a thioredoxin protein or peptide having a thioredoxin active site in a reduced state effective to decrease the viscoelasticity of the mucus or sputum as compared to prior to the step of contacting, wherein the thioredoxin protein or peptide comprises deletion and/or substitution and/or combination thereof of one or more cysteine residues outside of the thioredoxin active site with any amino acid residue other than a cysteine, and preferably when all non-active site cysteines are modified to other non-cysteine amino acids.

According to the present invention, the term “mucus” generally refers to a usually clear viscid fluid that is secreted by mucous membranes in various tissues of the body, including by the respiratory, gastrointestinal, and reproductive tracts. Mucus moistens, lubricates and protects the tissues from which it is secreted. It comprises mucin macromolecules (including mucus proteins, nucleic acids and carbohydrates), which are the gel-forming constituents of mucus. Mucus proteins include but are not limited to respiratory mucus proteins, digestive tract mucus proteins, reproductive tract mucus proteins, and ocular mucus proteins. The viscoelastic properties of normal mucus are dependent on the concentration, molecular weight, and degree of entanglement between mucin polymers. The term “sputum” generally refers to a mixture of saliva and discharge from the respiratory passages, including mucus. Sputum is typically an expectorated mixture of saliva and mucus (and other discharge from the respiratory tissues). Therefore, mucus is a primary component of sputum, and as such, the presence of excessively viscoelastic mucus results in a sputum which is itself excessively viscoelastic. The present invention relates to decreasing the viscosity and/or stiffness of abnormally viscoelastic mucus or sputum.

The term “liquefaction” refers to the act of becoming more liquid. Therefore, an increase in the liquefaction of mucus or sputum refers to the increase in liquid phase or liquid state of mucus or sputum, as compared to a more solid or viscous phase. In the case of abnormally viscous or excessive mucus associated with disease, the objective is to restore a normal level of mucus viscosity. Hence, liquefaction (or “normalization”) may also be considered as a reduction in mucus viscosity. Excessive liquefaction is itself deleterious so it is particularly desirable for liquefying agents to naturally limit their activity so that mucus is normalized rather than being liquefied completely.

It is appreciated that normal mucus function is achieved by having the appropriate ratio of biological reductants to oxidizable cysteines. Hence, a deficiency of biological reductant activity is therefore caused by either an excess of oxidized cysteines or a lack of biological reductants. Restoration of appropriate levels of biological reduction activity is therefore a means of ensuring the correct balance between oxidation (disulfide bond formation) and reduction (disulfide bond cleavage) when either oxidative stresses are increased and/or mucus levels are elevated or natural reductant activity levels are decreased.

The general functions of mucus and sputum in the body require that the mucus (and thus the mucus component of the sputum) have viscoelastic properties. In an individual with normal mucus and sputum (i.e., a healthy individual, or more particularly, an individual who does not suffer from symptoms or a condition caused or exacerbated by the viscosity or cohesiveness of mucus or sputum), the viscoelasticity is dependent on the concentration, molecular weight, and entanglements between mucin polymers (Verdugo et al., Biorheology 20:223-230, 1983). Especially in CF, when mucins in the mucus interact with DNA and f-actin polymers released from dying inflammatory cells, the mucus (and thus sputum) can additionally become even more dense and viscous. The inability to clear abnormal, thickened mucus by cough or mucociliary clearance facilitates colonization of the lung with opportunistic pathogens.

Therefore, abnormally or excessively viscous and/or cohesive mucus is characterized as mucus that is measurably or detectably more viscous or cohesive than mucus from a normal or healthy patient (preferably an age and sex-matched patient), and/or as mucus which, by virtue of its level of viscosity and/or cohesiveness, causes or contributes to at least one symptom in a patient that causes discomfort or pain to the patient, or that causes or exacerbates a condition or disease. In other words, abnormally or excessively viscous and/or cohesive sputum is a deviation from normal mucus or sputum wherein it is desirable to treat the patient to provide some relief from the condition or other therapeutic benefit. The abnormal mucus can be mobile secreted mucus as in the case of the airway surface, or static secreted mucus as in the gastrointestinal tract, buccal and nasopharyngeal cavities, reproductive tract, or the eye.

The methods and compositions of the present invention can be used to treat any patient in whom it is desirable to decrease the viscoelasticity of mucus or sputum as well as for the treatment of inflammation, hypertension, fibrosis, oxidative stress or infection and more preferably wherein the thioredoxin protein or peptide is administered by infusion or injection. Patients that have certain lung, sinus, nasal, ocular, digestive or gastrointestinal, or reproductive diseases or conditions can benefit from treatment using the methods and compositions of the present invention.

The present invention is most useful for ameliorating or reducing at least one symptom of a condition or disease that is caused by or exacerbated by abnormal or excessive viscoelasticity and/or cohesiveness of the mucus or sputum, which of course can include lung-associated diseases such as cystic fibrosis, as well as digestive diseases, such as coccidiosis or inflammatory bowel disease where abnormally viscoelastic mucus may be combined with inflammation and impaired response to pathogens.

Other diseases may, at least some of the time, be associated with abnormal or excessive viscoelasticity and/or cohesiveness of the mucus or sputum, and when such a symptom occurs, the method of the present invention can be used to decrease viscoelasticity of the mucus or sputum and provide at least some relief or therapeutic benefit to the patient. Examples of such diseases include, but are not limited to: cystic fibrosis; chronic or acute bronchitis; bronchiectasis (non-CF and CF bronchiectasis); COPD/emphysema; acute tracheitis (bacterial, viral, mycoplasmal or caused by other organisms); acute or chronic sinusitis; atelectasis (lung or lobar collapse) resulting from acute or chronic mucus plugging of the airways (sometimes seen in a variety of diseases such as asthma, including status asthmaticus); bronchiolitis (viral or other); acute, subacute or chronic bowel obstruction due to mucus inspissation including, but not limited to meconium ileus or meconium ileus equivalent in CF or similar disorders; other digestive diseases and infertility due to obstruction of (but not limited to) the cervix, seminal ducts or other vital reproductive structures, and dry-eye disease where abnormally thickened mucus secretions promote a vicious cycle of inflammation and further abnormal secretions. In addition, as improved mucociliary clearance is associated with clearance of bacteria and other pathogens from the lung, the composition and method of the present invention may be useful for reducing symptoms associated with excessive viscoelasticity and/or cohesiveness of the mucus or sputum in patients with a variety of respiratory infections, including both viral and bacterial infections.

Thioredoxin has a role in modulating runaway inflammatory responses and acute lung injury. Extracellular thioredoxin acts broadly to lower inflammation in animals subject to ongoing inflammatory processes. This has been observed in mouse models of COPD where neutrophilic inflammation was inhibited by thioredoxin, and in models of acute lung injury induced by influenza A virus infection where exogenous delivery or transgenic overexpression of thioredoxin prevented viral pneumonia in mice. Thioredoxin was found to suppress induction of the pro-inflammatory mediators TNF-a and CXCL1 in lavage fluid and lung tissue in mice in vivo, and in murine lung epithelial cells in vitro. In mice, thioredoxin inhibited lipopolysaccharide-induced neutrophil chemotaxis and LPS-induced IL-1b expression in human macrophages. The anti-inflammatory and immune-modulatory effects of thioredoxin have been proposed to involve control of cytokine mediator release, suppression of intercellular adhesion molecule-1 (ICAM-1) expression, and inhibition of inflammasome activity. Importantly, thioredoxin also acts to protect airway AT2 stem cells from inflammatory damage. These effects are likely exerted via allosteric control mechanisms as well as by direct activity on inflammatory targets. However, rapid clearance and poor pharmacology were found to be significant functional limitations for therapeutic use of exogenous native thioredoxin. Also, high concentrations of thioredoxin in the cell nucleus may paradoxically result in pro-inflammatory cytokine release in response to stimuli.

Accordingly, one embodiment of the present invention relates to a method of treating lung inflammation, runaway inflammatory responses and acute lung injury such as those associated with a viral respiratory disease. Such methods include administering a composition comprising a protein or peptide comprising a thioredoxin monocysteinic active site in a reduced state to a subject having or at risk of developing such conditions and/or a viral respiratory disease. The protein or peptide with a monocysteinic active site can be any described herein which are inhaled, topical anti-inflammatory and mucus-normalizing therapeutics. Such therapeutic compositions are believed to provide compartmentalization of activity to prevent intracellular/nuclear reductive stress and improve pharmacokinetics compared to a native thioredoxin.

In this embodiment, lung inflammation, runaway inflammatory responses and acute lung injury can be associated with a viral respiratory disease which is an illness caused by a virus and affects the respiratory tract. Such viral respiratory diseases can include Acute Respiratory Distress Syndrome (ARDS), Severe Acute Respiratory Distress Syndrome (SARS), Middle East Respiratory Syndrome (MERS), SARS-Coronavirus-2 (SARS-CoV-19 or COVID-19), influenza, viral infection associated with asthma, pneumonia, bronchitis, tuberculosis, reactive airway disease syndrome, and interstitial lung disease. The viruses involved that can cause one or more viral respiratory diseases including coronaviruses, influenza viruses, respiratory syncytial virus (RSV), parainfluenza viruses, and respiratory adenoviruses.

Emerging evidence strongly implicates the SARS-CoV-2 infected respiratory epithelium in initiation of the cascade of events that can lead to severe COVID-19 disease. In these severely-affected individuals dysregulation of airway cytokine release following infection can result in cytokine release syndrome (CRS) where immune system hyper-reactivity triggers a runaway response to infection causing more damage than the pathogen itself. Alveolar epithelial type II (AT2) cells, the stem cells of the adult lung and also the cells that produce airway surfactant respond to pathogens and alveolar damage by secreting cytokines to signal recruitment and initiate activation of macrophages to defend the alveolus. When this response becomes abnormally activated it can lead to CRS, which in severely-affected patients becomes systemic leading to overwhelming, lethal pathology. However, loss of AT2 cells due to the direct cytotoxic effects of virus infection can also lead to impairment of respiratory function as the accumulation of dead cells prevents efficient mucociliary clearance and enhances lung fluid retention and pneumonia, resulting in a vicious cycle of increasing inflammatory response.

ARDS, one of the most dreaded complications of COVID-19 and severe influenza, is associated with widespread inflammation in the lungs. The underlying mechanism of ARDS involves diffuse injury to cells which form the barrier of the microscopic air sacs (alveoli) of the lung, surfactant dysfunction, and activation of the immune system. The fluid accumulation in the lungs associated with ARDS is partially explained by vascular leakage due to inflammation. An important aspect of ARDS, triggered by infection, is an initial release of chemical signals and other inflammatory mediators secreted by lung epithelial and endothelial cells. Neutrophils and some T-lymphocytes migrate into the inflamed lung tissue and contribute to the amplification/deterioration of ARDS. A decrease in the production of lipid mediators of inflammation (prostaglandins) may impair the resolution of inflammation associated with ARDS (Fukunaga, et. al., Cyclooxygenase 2 Plays a Pivotal Role in the Resolution of Acute Lung Injury. Journal of Immunology 2005; 174:5033-5039.; Gao et al J Immunol 2017; 199:2043-2054).

Further disease or conditions the method of the present invention can be used for is for treating inflammation, hypertension, oxidative stress, infection, or fibrosis. Thus, in some embodiments, the invention includes a method to treat a bacterial or other infection in a subject. In this embodiment, the method can include a composition formulated for administration to a patient by a route selected from the group consisting of oral, rectal, nasal, inhaled, intratracheal, bronchial, direct installation, topical, and ocular including ocular injection. In some embodiments for treatment of infection, the composition can be a purified pharmaceutical composition, a nutraceutical, or a crude or purified extract of microbial cells expressing the protein or peptide. Such extracts, for example, are useful for use in animal feed compositions. In some embodiments, the invention includes a composition that includes a thioredoxin monocysteinic active site operable to activate an antimicrobial peptide, wherein the activation results in a therapeutically effective reagent to treat or prevent infectious diseases. Such an antimicrobial peptide can be a defensin.

Other embodiments of the invention include a method to modulate the microbiome composition of a subject, including administering topically to a mucosal surface of the subject a composition comprising a protein or peptide comprising a thioredoxin monocysteinic active site in a reduced state. Such a mucosal surface can be a pulmonary surface, a nasopharyngeal surface, or a gastrointestinal surface. In such embodiments, modulation of the microbiome can be effected by a protein or peptide of the invention activating one or more antimicrobial peptides.

A therapeutic benefit is not necessarily a cure for a particular disease or condition, but rather, preferably encompasses a result which most typically includes alleviation of the disease or condition, elimination of the disease or condition, reduction or elimination of a symptom associated with the disease or condition, prevention or alleviation of a secondary disease or condition resulting from the occurrence of a primary disease or condition (e.g., infectious disease caused by opportunistic pathogenic microorganisms that take advantage of the excessively viscous mucus in the respiratory tract), and/or prevention of the underlying disease or condition, or a symptom associated with the disease or condition.

As used herein, the phrase “protected from a disease” refers to reducing the symptoms of the disease; palliative therapy (relieving or soothing a symptom of the disease without effecting a cure); reducing the occurrence of the disease, and/or reducing the severity of the disease or to alleviate disease at least one symptom, sign or cause of the disease or condition. Preventing refers to the ability of a composition of the present invention, when administered to a patient, to prevent a disease from occurring. Curing (or disease-modifying) refers to the ability of a composition of the present invention, when administered to a patient to cure the disease. To protect a patient from a disease includes treating a patient that has a disease (therapeutic treatment). Preventing a disease/condition includes preventing disease occurrence (prophylactic treatment). In particular, protecting a patient from a disease (or preventing disease) is accomplished by increasing (normalizing) the liquefaction of an abnormally viscous mucus or sputum in the patient by contacting the mucus or sputum with a thioredoxin protein or peptide as disclosed herein comprising a thioredoxin active site in a reduced state such that a beneficial effect is obtained. A beneficial effect can easily be assessed by one of ordinary skill in the art and/or by a trained clinician who is treating the patient.

The term “disease” refers to any deviation from the normal health of a patient and includes a state when disease symptoms are present, as well as conditions in which a deviation (e.g., infection, gene mutation, genetic defect, etc.) has occurred, but symptoms are not yet manifested.

Contact of the mucus and/or sputum of a patient with the thioredoxin protein or peptide in a reduced state as disclosed herein (or compositions comprising such a protein) is intended to result in decreased viscoelasticity/increased liquefaction of the mucus or sputum as compared to prior to contact with the composition. According to the present invention, a normalization of mucus or sputum can be any measurable or detectable increase in the level of liquefaction of mucus or sputum as compared to a prior level of liquefaction, and is preferably a statistically significant increase (i.e., differences in measured level of liquefaction between the patient sample and a baseline control are statistically significant with a degree of confidence of at least p<0.05).

Typically, the “baseline control” is a patient sample prior to the administration of the treatment, since normal, healthy individuals generally cannot produce a quantity of sputum sufficient to serve as a control, although sputum from a normal, healthy individual is not excluded as a baseline control. Additionally, a decrease in viscosity results in an improvement of lung function. This improvement can be determined by various means including patient reported outcomes, mean time of exacerbation to hospital admission and/or an increase in forced expiratory volume (FEV).

In one aspect of the invention, an increase in FEV is described as an increase of at least about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, and 9.5% and about 10% as compared to a sample from the patient prior to contact with a composition or protein of the present invention. Preferably, contact of a protein or composition of the present invention with the mucus or sputum of a patient sample results in an increase of about 2.5% as compared to a sample from the patient prior to contact with a composition or protein of the present invention.

Liquefaction of mucus or sputum and/or decrease in viscoelasticity can be measured using any suitable technique known in the art, including, but not limited to, compaction assays as described in the Examples section. In such an assay, the amount of mucus or sputum in a solid phase (gel) versus aqueous phase (liquid) is measured.

In other aspects of the invention, the relative viscosity or cohesiveness of mucus or sputum can be measured using other parameters or indicators including, but not limited to, viscoelasticity (measured, for example, by rheometry or magnetic microrheometry), glycoprotein content, or DNA content. In another aspect of the invention the change in mucus protein disulfide bonding can be estimated by the use of reagents such as NEM (N-Ethylmaleimide) that preferentially react with unbound (free) Cys residue thiol groups that are created by the disruption of disulfide bonds (Rancourt, R. et al., Free Radic Biol Med, 42(9):1441-1453, 2007).

In one aspect of the invention, the level of liquefaction is described as the amount of a given mucus or sputum sample that is in an aqueous (liquid) phase as a percentage of the total volume of the mucus or sputum sample. In a patient with cystic fibrosis, for example, the level of liquefaction of mucus or sputum can be as low as less than 10% or even less than 5% of the total volume. Preferably, contact of a protein or composition of the invention with the mucus or sputum results in a change in the liquefaction of the mucus or sputum such that at least about 15% of the total volume is in liquid phase, and more preferably, at least about 20% of the total volume is in liquid phase, and more preferably, at least about 25% of the total volume is in liquid phase, and more preferably, at least about 30% of the total volume is in liquid phase, and more preferably, at least about 35% of the total volume is in liquid phase, and more preferably, at least about 40% of the total volume is in liquid phase, and more preferably, at least about 45% of the total volume is in liquid phase, and more preferably, at least about 50% of the total volume is in liquid phase or until the blockage or inhibition of function caused by the mucus has cleared (e.g., until the patient airways are cleared sufficiently to begin expectorating the fluid). Increase beyond 80 or 90% is generally not desirable as complete liquefaction resulting in mucin depolymerization disrupts the beneficial viscoelasticity required for mucus transport via ciliary action. Excessive liquefaction of the mucus or sputum can also be detrimental to the patient (e.g., liquefied sputum could flow backward and flood the small airways with a thin liquid, that may also be infected, before the sputum can be cleared by the patient). In this regard, target-selective natural reductants such as thioredoxin are greatly preferred, as these have preference for highly structured disulfide bonds (e.g. as described in Passam, F. J., and Chiu, J., Allosteric disulphide bonds as reversible mechano-sensitive switches that control protein functions in the vasculature, Biophys Rev 11, 419-430, 2019) rather than planar disulfides that form the intermolecular bonds essential for creating the polymeric structure of mucus. Small molecule reducing agents lack target preference and hence can result in adverse effects due to over-liquefaction. In some embodiments, contact of a protein or composition of the invention with the mucus or sputum results in a change in the liquefaction of the mucus or sputum such that between about 15% and about 90% of the total volume is in liquid phase or any whole number range between 15% and 90%.

In general, it is therefore preferred that the liquefaction of the sputum or mucus in increased in small, gradual increments until the airway or other blocked passage (e.g., in the gastrointestinal or reproductive tract) is cleared, but without excessively liquefying the sputum. Preferably, the contact of a protein, peptide or composition of the invention with mucus or sputum produces at least about a 1% increase in the liquefaction of the mucus or sputum by volume as compared to prior to the treatment, more preferably, at least about a 2% increase, and so on, in increments of 1%, until the patient airways or other clogged passages are cleared. Once such clearing is attained, e.g. by removal of so-called “mucus plugs” to improve access of drug to the small airways and alveoli, then a lower-dose maintenance therapy may be undertaken in order to keep newly-secreted mucin proteins at a normal state of disulfide bonding. Thioredoxin, and in particular target-binding monothiol thioredoxin, is comparatively far less likely to create over-liquefaction than are non-selective reducing agents, greatly increasing the therapeutic window between effective and toxic doses.

In one aspect, the therapy is conducted in conjunction with methods to clear the thinned material from the affected tissue (respiratory tract, digestive tract, reproductive tract) of the patient. For example, in the case of the respiratory system, one can use the method of the present invention in conjunction with postural drainage, huff coughing and other respiratory exercises, or any other suitable method for expectorating the liquefied mucus or sputum.

According to the present invention, the mucus or sputum in the patient to be treated is contacted with a thioredoxin protein disclosed herein (or composition comprising the protein) that contains a substitution of one or more cysteine residues outside of the thioredoxin active site with any amino acid residue other than a cysteine. The protein is effective to reduce the viscoelasticity and cohesiveness of sputum or mucus and/or to increase the liquefaction of sputum or mucus as compared to prior to the step of contacting. As described previously, thioredoxin is a protein disulfide reductase found in most organisms that participates in many thiol-dependent cellular reductive processes. In humans, thioredoxin is also referred to as adult T cell leukemia-derived factor (ADF). Intracellularly, most of this ubiquitous low molecular weight (11,700) protein remains reduced. Reduced or oxidized thioredoxin may be able to enter intact cells or absorb to the cell membrane, where a small amount is gradually internalized over time. Native thioredoxin has two vicinal cysteine residues at the active site that in the oxidized protein form a disulfide bridge located in a protrusion from the protein's three-dimensional structure. The flavoprotein thioredoxin reductase catalyzes the NADPH-dependent reduction of this disulfide. In addition, engineered versions of thioredoxin reductase modified for altered cofactor specificity may utilize NADH instead or in addition to NADPH as described in U.S. Pat. No. 7,071,307, hereby incorporated by reference. Small increases in thioredoxin can cause profound changes in sulfhydryl-disulfide redox status in proteins. Oxidized thioredoxin, especially the secreted form, can also be reduced by the action of glutathione in conjunction with the secreted enzyme glutaredoxins (Du, Y., Zhang, H., Lu, J., and Holmgren, A., Glutathione and glutaredoxin act as a backup of human thioredoxin reductase 1 to reduce thioredoxin 1 preventing cell death by aurothioglucose, Journal of Biological Chemistry 287, 38210-38219, 2012). Both GSH and glutaredoxins are abundant in the airway.

In addition to its ability to effect the reduction of cellular proteins, it is recognized that thioredoxin can act directly as an antioxidant (e.g. by preventing oxidation of an oxidizable substrate by scavenging reactive oxygen species) as well as by activation of peroxidase enzymes, although, unlike other thiols, thioredoxin does not generally contribute to the oxidative stress in a cell by autooxidizing (e.g. generating superoxide radicals through autooxidation). U.S. Pat. No. 5,985,261 to White et al., supra, showed that thioredoxin directly induces the production of MnSOD and that such induction is effected by thioredoxin in a reduced state.

### Further Therapeutic Variants

In one embodiment, thioredoxin proteins or peptides as disclosed herein containing a thioredoxin active site can be products of drug design or selection and can be produced using various methods known in the art. Such proteins or peptides can be referred to as mimetics. A mimetic refers to any peptide or non-peptide compound that is able to mimic the biological action of a naturally-occurring peptide, often because the mimetic has a basic structure that mimics the basic structure of the naturally-occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example.

Such mimetics can be designed, selected and/or otherwise identified using a variety of methods known in the art. Various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety. Thioredoxin mimetic peptides capable of potent and selective redox activity are described by Bachnoff et al., Free Radical Biol Med 50:1355-67 (2011) and incorporated herein by reference in its entirety. A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

Diversity-creation methods such as the foregoing can be combined with other techniques designed to improve function or pharmacology, especially for reduced-size molecules like active-site mimetics. For example, one approach that has shown promise in early-stage studies is hydrocarbon-stapled a-helical peptides, a novel class of synthetic miniproteins locked into their bioactive a-helical fold through the site-specific introduction of a chemical brace, an all-hydrocarbon staple. Stapling can greatly improve the pharmacologic performance of peptides, increasing their target affinity and proteolytic resistance, while creating smaller peptide versions of larger proteins/enzymes that are suitable for chemical synthesis (Verdine, G. L. and Hilinsky, G. J., Methods Enzymol, 503:3-33, 2012).

In one embodiment of the present invention, a thioredoxin protein suitable for use in the present invention has an amino acid sequence that comprises, consists essentially of, or consists of a full length sequence of a thioredoxin protein or any fragment thereof that has a thioredoxin active site as described herein. For example, any one of the native sequences of SEQ ID NOs 4-15 or a fragment or other homologue thereof that contains a thioredoxin active site as described herein is encompassed by the invention. Such homologues can include proteins having an amino acid sequence that is at least about 10% identical to the amino acid sequence of a full-length thioredoxin protein, or at least 20% identical, or at least 30% identical, or at least 40% identical, or at least 50% identical, or at least 60% identical, or at least 70% identical, or at least 80% identical, or at least 90% identical, or greater than 95% identical to the amino acid sequence of a full-length thioredoxin protein, including any percentage between 10% and 100%, in whole integers (10%, 11%, 12%, . . . 98%, 99%, 100%).

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schaeffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

A protein useful in the present invention can also include thioredoxin proteins having an amino acid sequence comprising at least 10 contiguous amino acid residues of any full-length thioredoxin protein containing an active site (native sequences represented by SEQ ID NOs:4-15, i.e., 10 contiguous amino acid residues having 100% identity with 10 contiguous amino acids of a reference sequence) and having deletions and/or substitutions of the non-active cysteine residues outside of the active site. In other embodiments, a homologue of a thioredoxin protein includes amino acid sequences comprising at least 15, or at least 20, or at least 25, or at least 30, or at least 35, or at least 40, or at least 45, or at least 50, or at least 55, or at least 60, or at least 65, or at least 70, or at least 75, or at least 80 contiguous amino acid residues of the amino acid sequence of a naturally occurring thioredoxin protein, and so on, up to the full-length of the protein, including any intervening length in whole integers (10, 11, 12, . . . ) and which comprises an active site.

According to the present invention, the term “contiguous” or “consecutive”, with regard to sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In another embodiment, a protein useful in the present invention includes a thioredoxin protein having an amino acid sequence that is sufficiently similar to a natural thioredoxin amino acid sequence that a nucleic acid sequence encoding the homologue is capable of hybridizing under moderate, high or very high stringency conditions (described below) to (i.e., with) a nucleic acid molecule encoding the natural thioredoxin protein (i.e., to the complement of the nucleic acid strand encoding the natural thioredoxin amino acid sequence). Such hybridization conditions are described in detail below.

A nucleic acid sequence complement of a nucleic acid sequence encoding a thioredoxin protein of the present invention refers to the nucleic acid sequence of the nucleic acid strand that is complementary to the strand that encodes thioredoxin. It will be appreciated that a double-stranded DNA which encodes a given amino acid sequence comprises a single strand DNA and its complementary strand having a sequence that is a complement to the single strand DNA. As such, nucleic acid molecules of the present invention can be either double-stranded or single-stranded, and include those nucleic acid molecules that form stable hybrids under stringent hybridization conditions with a nucleic acid sequence that encodes an amino acid sequence of a thioredoxin protein, and/or with the complement of the nucleic acid sequence that encodes such amino acid sequence. Methods to deduce a complementary sequence are known to those skilled in the art.

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides).

As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids.

In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tm can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tm of a particular hybrid, and wash conditions typically include a combination of salt and temperature conditions that are approximately 12-20° C. below the calculated Tm of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

Fusions of Thioredoxin with Various Sequences

A thioredoxin protein of the present invention can also be a fusion protein that includes a segment containing a thioredoxin active site and a fusion segment that can have a variety of functions. For example, such a fusion segment can function as a tool to simplify purification of a protein of the present invention, such as to enable purification of the resultant fusion protein using affinity chromatography. A suitable fusion segment can be a domain of any size that has the desired function (e.g., imparts increased stability to a protein, imparts increased immunogenicity to a protein, and/or simplifies purification of a protein). It is within the scope of the present invention to use one or more fusion segments. Fusion segments can be joined to amino and/or carboxyl termini of the segment containing a thioredoxin active site. Linkages between fusion segments and thioredoxin active site-containing domains of fusion proteins can be susceptible to cleavage in order to enable straightforward recovery of the thioredoxin active site-containing domains of such proteins. Fusion proteins are preferably produced by culturing a recombinant cell transformed with a fusion nucleic acid molecule that encodes a protein including the fusion segment attached to either the carboxyl and/or amino terminal end of a thioredoxin active site-containing domain.

In one embodiment of the present invention, any of the amino acid sequences described herein, such as the amino acid sequence of a naturally occurring thioredoxin protein or thioredoxin containing an active site, can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally-occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

### Sources of Thioredoxin

In one embodiment, a thioredoxin protein or peptide as disclosed herein containing a thioredoxin active site suitable for use with the method of the present invention comprises a protein or peptide containing a thioredoxin active site derived from a substantially similar species of animal as that to which the protein is to be administered. In another embodiment, any thioredoxin protein or peptide as disclosed herein containing a thioredoxin active site, including from diverse sources such as microbial, plant and fungus can be used in a given patient.

In another embodiment, a thioredoxin protein or peptide as disclosed herein containing a thioredoxin active site suitable for use with the method of the present invention comprises an isolated, or biologically pure, protein. As such, “isolated” and “biologically pure” do not necessarily reflect the extent to which the protein has been purified. An isolated protein of the present invention can, for example, be obtained from its natural source, be produced using recombinant DNA technology (e.g., polymerase chain reaction (PCR) amplification, cloning), or be synthesized chemically.

In yet another embodiment, a chemically-synthetic thioredoxin protein or peptide containing a thioredoxin active site of the present invention may also refer to a stabilized version, such as one containing an active site constrained structurally by stapled peptide technology, by cyclization, or by constraint at the N or C termini. Preferably, the thioredoxin protein containing a thioredoxin active site to be used in methods of the invention have a half-life in vivo that is sufficient to cause a measurable or detectable increase in liquefaction (or decrease in the viscosity or cohesiveness) of mucus or sputum in a patient, and or to cause a measurable, detectable or perceived therapeutic benefit to the patient that is associated with the mucus and sputum in the patient. Such half-life can be effected by the method of delivery of such a protein. A protein of the present invention preferably has a half-life of greater than about 5 minutes in an animal, and more preferably greater than about 4 hours in an animal, and even more preferably greater than about 16 hours in an animal. In a preferred embodiment, a protein of the present invention has a half-life of between about 5 minutes and about 24 hours in an animal, and preferably between about 2 hours and about 16 hours in an animal, and more preferably between about 4 hours and about 12 hours in an animal.

### Nucleic Acid Molecules Related to Thioredoxins

Further embodiments of the present invention include nucleic acid molecules that encode a thioredoxin protein or peptide as disclosed herein containing a thioredoxin active site. Such nucleic acid molecules can be used to produce a protein that is useful in the method of the present invention in vitro or in vivo. A nucleic acid molecule of the present invention includes a nucleic acid molecule comprising, consisting essentially of, or consisting of, a nucleic acid sequence encoding any of the proteins described previously herein. In accordance with the present invention, an isolated nucleic acid molecule is a nucleic acid molecule (polynucleotide) that has been removed from its natural milieu (i.e., that has been subject to human manipulation) and can include DNA, RNA, or derivatives of either DNA or RNA, including cDNA. As such, “isolated” does not reflect the extent to which the nucleic acid molecule has been purified. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein.

An isolated nucleic acid molecule of the present invention can be isolated from its natural source or produced using recombinant DNA technology (e.g., polymerase chain reaction (PCR) amplification, cloning) or chemical synthesis. Isolated nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode the desired protein of the present invention or to form stable hybrids under stringent conditions with natural gene isolates. An isolated nucleic acid molecule can include degeneracies. As used herein, nucleotide degeneracies refers to the phenomenon that one amino acid can be encoded by different nucleotide codons. Thus, the nucleic acid sequence of a nucleic acid molecule that encodes a given protein useful in the present invention can vary due to degeneracies.

According to the present invention, reference to a gene includes all nucleic acid sequences related to a natural (i.e., wildtype) gene as well as those related to the thioredoxin monocysteinic active site, such as regulatory regions that control production of the protein encoded by that gene (such as, but not limited to, transcription, translation or post-translation control regions) as well as the coding region itself. In another embodiment, a gene can be a naturally occurring allelic variant that includes a similar but not identical sequence to the nucleic acid sequence encoding a given protein. Allelic variants have been previously described above. The phrases “nucleic acid molecule” and “gene” can be used interchangeably when the nucleic acid molecule comprises a gene as described above.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on protein biological activity. Allelic variants and protein homologues (e.g., proteins encoded by nucleic acid homologues) have been discussed in detail above.

A nucleic acid molecule homologue can be produced using a number of methods known to those skilled in the art (e.g., as described in Sambrook et al., ibid). For example, nucleic acid molecules can be modified using a variety of techniques including, but not limited to, by classical mutagenesis and recombinant DNA techniques (including without limitation site-directed mutagenesis, chemical treatment, restriction enzyme cleavage, ligation of nucleic acid fragments and/or PCR amplification), or synthesis of oligonucleotide mixtures and chemical ligation, or in vitro or in vivo recombination, of mixtures of molecular groups to “build” a re-assorted library of nucleic acid molecules comprising a multiplicity of combinations thereof by the process of gene shuffling (i.e., molecular breeding; see, for example, U.S. Pat. No. 5,605,793 to Stemmer; Minshull and Stemmer, Curr. Opin. Chem. Biol. 3:284-290, 1999; Stemmer, P.N.A.S. USA 91:10747-10751, 1994, all of which are incorporated herein by reference in their entirety). These and other similar techniques known to those skilled in the art can be used to efficiently introduce multiple simultaneous changes in the protein. Nucleic acid molecule homologues can subsequently be selected by hybridization with a given gene, or be screened by expression directly for function and biological activity of proteins encoded by such nucleic acid molecules.

One embodiment of the present invention relates to a recombinant nucleic acid molecule that comprises the isolated nucleic acid molecule described above which is operatively linked to at least one transcription control sequence. More particularly, according to the present invention, a recombinant nucleic acid molecule typically comprises a recombinant vector and the isolated nucleic acid molecule as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is, nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a replicating plasmid) or it can be integrated into the chromosome of a recombinant host cell, although it is preferred if the vector remain separate from the genome for most applications of the invention. The entire vector can remain in place within a host cell, or under certain conditions, the plasmid DNA can be deleted, leaving behind the nucleic acid molecule of the present invention. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced (e.g., the protein containing a thioredoxin monocysteinic active site) is inserted into the recombinant vector to produce a recombinant nucleic acid molecule. The nucleic acid sequence encoding the protein to be produced is inserted into the vector in a manner that operatively links the nucleic acid sequence to regulatory sequences in the vector that enable the transcription and translation of the nucleic acid sequence within the recombinant host cell.

In another embodiment of the invention, the recombinant nucleic acid molecule comprises a viral vector. A viral vector includes an isolated nucleic acid molecule of the present invention integrated into a viral genome or portion thereof, in which the nucleic acid molecule is packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

Typically, a recombinant nucleic acid molecule includes at least one nucleic acid molecule of the present invention operatively linked to one or more expression control sequences. As used herein, the phrase “recombinant molecule” or “recombinant nucleic acid molecule” refers primarily to a nucleic acid molecule or nucleic acid sequence operatively linked to an expression control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence in a manner such that the molecule is able to be expressed when transfected (i.e., transformed, transduced, transfected, conjugated or conduced) into a host cell.

Transcription control sequences are expression control sequences that control the initiation, elongation, or termination of transcription. Particularly important transcription control sequences are those that control transcription initiation, such as promoter, enhancer, operator and repressor sequences. Suitable transcription control sequences include any transcription control sequence that can function in a host cell or organism into which the recombinant nucleic acid molecule is to be introduced. Recombinant nucleic acid molecules of the present invention can also contain additional regulatory sequences, such as translation regulatory sequences, origins of replication, and other regulatory sequences that are compatible with the recombinant cell.

In one embodiment, a recombinant molecule of the present invention, including those that are integrated into the host cell chromosome, also contains secretory signals (i.e., signal-segment or signal-sequence nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention.

In another embodiment, a recombinant molecule of the present invention comprises a leader sequence to enable an expressed protein to be delivered to and inserted into the membrane of a host cell. Other signal sequences include those capable of directing periplasmic or extracellular secretion, or retention within desired compartments. Suitable leader sequences include a leader sequence that is naturally associated with the protein, or any heterologous leader sequence capable of directing the delivery and insertion of the protein to the membrane of a cell.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells or plants. In microbial systems, the term “transformation” is used to describe an inherited change due to the acquisition of exogenous nucleic acids by the microorganism and is essentially synonymous with the term “transfection.” However, in animal cells, transformation has acquired a second meaning which can refer to changes in the growth properties of cells in culture (described above) after they become cancerous, for example. Therefore, to avoid confusion, the term “transfection” is preferably used with regard to the introduction of exogenous nucleic acids into animal cells, and is used herein to generally encompass transfection of animal cells and transformation of plant cells and microbial cells, to the extent that the terms pertain to the introduction of exogenous nucleic acids into a cell. Therefore, transfection techniques include, but are not limited to, transformation, particle bombardment, electroporation, microinjection, lipofection, adsorption, infection and protoplast fusion.

### Administration to Human and Non-human Vertebrates

In the methods of the present invention, compositions, including pharmaceutical compositions can be administered to patients of any member of the Vertebrate class, including, without limitation, primates, rodents, livestock, chickens, turkeys and domestic pets, companion animals, or racehorses.

As discussed above, a composition, including a pharmaceutical composition, of the present invention is administered to a patient in a manner effective to deliver the composition, and particularly the thioredoxin protein as disclosed herein comprising a thioredoxin active site and/or any other compounds in the composition, to a target site (e.g., mucus or sputum to be treated for proteins and compounds, a target host cell that will be or is in the environment of the mucus or sputum to be treated for recombinant nucleic acid molecules). Suitable administration protocols include any in vivo or ex vivo administration protocol.

According to the present invention, an effective administration protocol (i.e., administering a composition of the present invention in an effective manner) comprises suitable dose parameters and modes of administration that result in contact of the thioredoxin protein disclosed herein containing a thioredoxin active site and/or other compound in the composition with the mucus or sputum to be treated, preferably so that the patient obtains some measurable, observable or perceived benefit from such administration. Alternatively, effective dose parameters can be determined by experimentation using in vitro samples, in vivo animal models, and eventually, clinical trials if the patient is human. Effective dose parameters can be determined using methods standard in the art for a particular disease or condition. Such methods include, for example, determination of survival rates, side effects (i.e., toxicity) and progression or regression of disease, as well as relevant physiological parameters such as forced expiratory volume in one second (FEV, FEV1).

According to the present invention, suitable methods of administering a composition of the present invention to a patient include any route of in vivo administration that is suitable for delivering the composition to the desired site in or on a patient. The preferred routes of administration will be apparent to those of skill in the art, depending on whether the compound is a protein or other compound (e.g., a drug), to what part of the body the composition is to be administered, and the disease or condition experienced by the patient. In general, suitable methods of in vivo administration of a thioredoxin protein or peptide as disclosed herein include, but are not limited to, dermal delivery, intratracheal administration, inhalation (e.g., aerosol), nasal, oral, pulmonary administration, and impregnation of a catheter. Aural delivery can include ear drops, intranasal delivery can include nose drops or intranasal injection, and intraocular delivery can include eye drops or the use of suitable devices for passage of the drug across the sclera and/or to the back of the eye. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can include solids and liquids that can be taken through the mouth, for example, as tablets or capsules, as well as being formulated into food and beverage products or animal feed or feed pellets. Other routes of administration that are useful for mucosal tissues include bronchial, intranasal, other inhalatory, rectal, topical, transdermal, vaginal, transcervical, pericervical and urethral routes. In addition, administration protocols can include pretreatment devices, such as application of the protein, peptide or composition in a diaphragm (e.g., to the cervix) for use in applications such as infertility, and surgical-assisted topical administration such as injection into the sinus cavities.

In a preferred embodiment of the present invention, when the protein or composition of the invention is administered to treat excessively or abnormally viscous or cohesive sputum or mucus in the respiratory tract (airways), a protein or peptide (or composition) containing a thioredoxin monocysteinic active site or other compound is administered by a route including, but not limited to, inhalation (i.e. by inhaling an aerosol, e.g., in or with surfactants); direct installation into the lung via a bronchoscope, endotracheal tube and/or via any artificial ventilation device; nasal administration (intranasal or transnasal), bronchial, or intratracheally (i.e. by injection directly into the trachea or tracheostomy), either directly or via lipid-encapsulation or surfactant. Any conceivable method of introducing the composition or protein into the airways so that it can contact the mucus or sputum therein is encompassed by the invention.

### Feed

Another embodiment of the present invention relates to an animal feed composition comprising a thioredoxin protein or peptide as disclosed herein containing a thioredoxin active site in a reduced state.

Animal feed is used to meet the nutritional requirements of domesticated animals of any type. Animal feed encompasses both fodder and forage. For example, a thioredoxin protein or peptide disclosed herein can be used in or on fodder and/or forage by mixing into or with, applying to, or incorporating by any means into or onto fodder and/or forage. Examples of animal feed include but are not limited to hay, straw, silage, compressed and pelleted feeds, oils and mixed rations, sprouted grains, legumes, crop residue, grain, cereal crop, and corn.

Animal feed encompasses feed for companion animals, livestock, and other types of animals for which it is desired to meet nutritional requirements. Companion animals include but are not limited to dogs, cats, other mammals, birds, reptiles, amphibians, fish, and other companion animals. Livestock includes but is not limited to cows, horses, buffalo, sheep, goats, pigs, other ungulates, chickens, turkeys, ducks, other birds, salmon, trout, carp, tilapia, catfish, other fish, or other types of livestock. The thermal stability characteristics of monothiol thioredoxin make it particularly amenable for incorporation into pelleted feeds that must withstand heating in excess of 80 degrees C. for several minutes.

Each of the publications and other references discussed or cited herein is incorporated herein by reference in its entirety.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

### EXAMPLES

For the examples below, “ORP100S” is provided as an exemplary thioredoxin protein and any of the thioredoxin proteins disclosed herein can substitute for ORP100S.

### Example 1. ORP100S Expression and Characterization

This example demonstrates the structure, construction, expression and evaluation of ORP100S. Compared to the C35S monocysteinic active site thioredoxin ORP-100, ORP100S additionally incorporates mutation to Ser of the three remaining non-active site thioredoxin-1 Cys residues, resulting in a fully monocysteinic thioredoxin with improved stability, activity and more robust analytics.

### Monothiol C35S Active Site Thioredoxins ORP-100 and ORP100S

The active site of the native Trx enzyme contains two redox-active Cys residues that are highly conserved across species. In their inactive oxidized form, these Cys constitute a disulfide bridge that protrudes from the three-dimensional structure of the protein (Holmgren A., 1985, Thioredoxin, Annu Rev Biochem 54:237-71). Reduction of this active center (by the TrxR enzyme, GSH/glutarodoxin, or via synthetic activation with chemical reductants) allows Trx to function as an electron carrier with dithiol/disulfide exchange capability. Protein disulfides are a preferred substrate for Trx-mediated reducing activity. Initially, a transient mixed-disulfide is formed between the N-terminal Cys32 of the thioredoxin active-site and a Cys of a compatible target disulfide following nucleophilic attack by the Cys32 thiolate anion (Holmgren A., 1995, Thioredoxin structure and mechanism: conformational changes on oxidation of the active-site sulfhydryls to a disulfide. Structure 3:239-43). In native Trx the C-terminal active site cysteine (Cys35) then becomes activated due to conformational change in the active site which stabilizes the Cys35 thiolate anion, dropping the pKa and allowing attack on the intramolecular mixed disulfide linkage resulting in release of oxidized Trx and a now fully-reduced target (Wynn R, Cocco M J, Richards F M., 1995, Mixed disulfide intermediates during the reduction of disulfides by Escherichia coli thioredoxin. Biochemistry 34:11807-13).

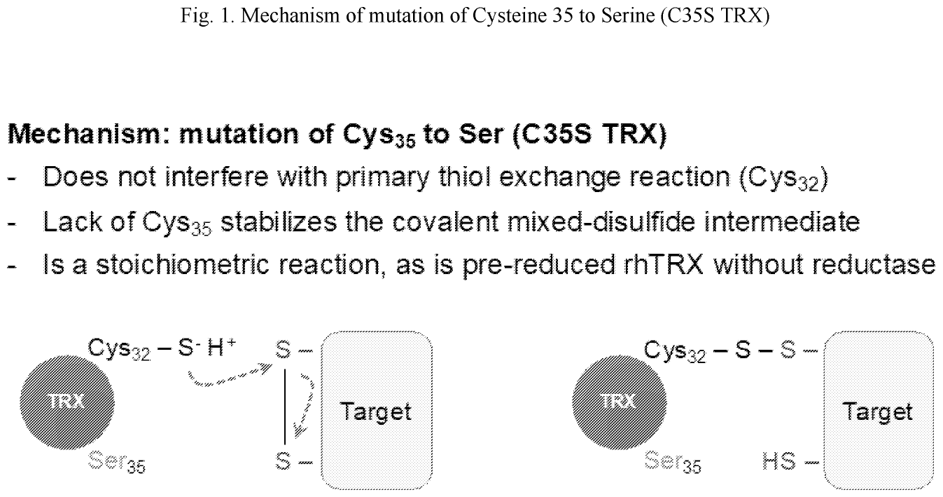

ORP-100 and ORP100S are modified versions of Trx that have been engineered by mutation of the active site Cys35 to Ser (C35S Trx). As illustrated in FIG. 1, this eliminates the second stage of the Trx-disulfide reduction by preventing resolution of the mixed-disulfide intermediate formed by the primary Cys32 reaction and results in a stable, covalent linkage of this Cys to the protein target. In the case of human CF mucus, treatment with reduced C35S Trx disrupts excessive disulfide bonds in condensed mucin proteins to normalize mucus viscosity, while the Trx-mucin adduct blocks the ability of new mucin Cys disulfides to re-form. In addition, the covalent linkage to mucus immobilizes the C35S Trx enzyme extracellularly and prevents cellular uptake. This unique blockade mechanism allows the modified Trx to act appropriately on the mucosal surface but reduces or eliminates the chance for activation of inflammatory pathways or other off-target effects that might be induced by Trx signaling within cells. Crucially, this monothiol active-site C35S Trx strategy for the first time enables replacement or replenishment of the activity of secreted Trx without markedly affecting intracellular Trx activity.

ORP100S vs. ORP-100

Compared to ORP-100, ORP100S has been further modified by Cys-to-Ser mutation of the remaining non active-site Trx Cys residues located at positions 62, 69 and 73. The rationale for this was twofold: 1) to eliminate reactive Cys capable of mediating protein:protein interactions and homodimerization/multimerization that could result in decreased availability of functional protein and increased instability of the fully reduced monomeric C35S thioredoxin; and 2) to enable the use of Cys redox state quantification as a robust and simple in-process assay to monitor overall protein reduction level and catalytic activity potential. The three non active-site Cys do not serve structural functions in native Trx and are thought to play primarily regulatory roles via the formation of intermolecular linkages that attenuate disulfide bond reducing activity, including homodimerization at Cys73 (Weichsel, A., Gasdaska, J. R., Powis, G., and Montfort, W. R., 1996, Crystal structures of reduced, oxidized, and mutated human thioredoxins: evidence for a regulatory homodimer. Structure 4, 735-51). Non active-site Trx Cys have also been shown to be sites for S-nitrosylation by GSNOR and potentially other post-translational modifications (Wu C, Liu T, Chen W, et al. Redox regulatory mechanism of transnitrosylation by thioredoxin, 2010, Molecular & Cellular Proteomics 9:2262-75). Oxidative stability of ORP100S, a fully monocysteinic Trx, is also increased by elimination of the potential for multimerization, as only dimerization at Cys32 is possible in ORP100S vs. the potential for multiple dimer and higher multimeric forms in C35S Trx variants that also retains one or more of the non active-site Cys.

Since the reduced thiol of any of the Trx Cys is capable of reducing a chromogenic substrate such as DTNB (5,5′-dithiobis-(2-nitrobenzoic acid) to induce a quantifiable absorbance change but only Cys32 is able to form a mixed-disulfide with an appropriate protein disulfide substrate such as insulin, removal of all Cys except Cys32 also means that the reduction state of the total Cys in ORP100S is identical to the reduction state of Cys32. Hence, the activity of ORP100S to reduce DTNB is the same as its ability to reduce a protein disulfide bond. This allows spectrophotometric monitoring of DTNB reduction to be used as a direct measure of ORP100S protein activity rather than the more complicated and time-consuming determination of insulin reduction state using reverse-phase high-performance liquid chromatography (RP-HPLC).

### ORP100S Design and Construction

The sequence of ORP100S was codon-optimized for expression in E. coli with a custom algorithm based on the amino acid sequence of human thioredoxin-1. This was hypothesized to both increase expression level and prevent amino acid misincorporation resulting from depletion of tRNAs less common in bacteria vs. humans, both of which are significant challenges for recombinant expression of native eukaryotic thioredoxin gene sequences in E. coli (Harris et a., 2012, Determination and control of low-level amino acid misincorporation in human thioredoxin protein produced in a recombinant Escherichia coli production system. Biotechnology and Bioengineering 109, 1987-95).

ORP100S was synthesized as a DNA fragment flanked by 011 and HindIII restriction sites for convenient manipulation and cloned into expression vector pD861 (DNA2.0/Atum) under control of a rhamnose-inducible promoter, and transformed into BL21 E. coli. In some strains the E. coli rhaB gene was deleted to enhance rhamnose induction. Rhamnose-inducible expression was verified by SDS-polyacrylamide gel electrophoresis (SDS-PAGE) following growth at small scale in 2 ml volume culture blocks.

### ORP100S Expression, Purification and Analysis

The initial strategy for benchtop scale production of ORP100S was the following: cells were grown under fed-batch conditions in 1.5L fermenters (Dasgip, Eppendorf) and cell paste recovered by centrifugation prior to disruption and primary recovery by ultrafiltration. ORP100S protein was purified to >95% by anion exchange chromatography followed by size-exclusion fast protein liquid chromatography (SEC-FPLC) and ultrafiltration/diafiltration. For activation (reduction), ORP100S was treated with 10 mM dithiothreitol (DTT) then exchanged into lyophilization buffer with an endotoxin clearance step to remove DTT and endotoxins. Reduced proteins were frozen at −80° C. and lyophilized (Virtis) for 24-36 hours. Sequence identity and homogeneity was verified by MALDI-TOF and ESI mass spectrometry following size confirmation and purity determination by SDS-PAGE and analytical SEC-HPLC. Color was slightly yellow resulting in an off-white lyophilizate.

Functional assays
1. Reduction state of ORP100S was quantified using DTNB which reacts with free SH groups resulting in a yellow color change at 412 nm. Fifty microliters of 2.5 mM rhTrx was added to a 96-well plate, followed by 175 microliters of sample buffer and 25 microliters of 6 mM DTNB. After reactions were initiated by the addition of DTNB, change in kinetic absorbance at 412 nm due to DTNB reduction was monitored spectrophotometrically at 30° C. ORP100S concentration was determined by A₂₈₀ (Nanodrop) using the extinction coefficient of human Trx-1 (7,000). The actual concentrations of free sulfhydryl groups were calculated based on the absorbance at 412 nm and the extinction coefficient of DTNB (14,150) in order to determine the reduction state as percentage of free sulfhydryl. For ORP100S this represents 100% of the protein disulfide reducing capacity whereas for ORP-100 with only one of four Cys capable of target reduction it represents 25%.
2. Percent of free monomer ORP100S in solution was determined using SEC. Samples were analyzed on a BioBasic S-300 250×4.6 column (Thermo Scientific) run on an Agilent 1100 HPLC system. The SEC-HPLC mobile phase buffer consisted of 40 mM ammonium acetate (pH 5.5), 2 mM EDTA and 450 mM NaCl. Low pH minimized dimerization while the 450 mM NaCl concentration improved resolution. The flow rate was 0.35 mL/min and absorbance was monitored at 280 nm. The length of each run was 20 minutes. The ORP100S monomer percentage was determined by integration of the area under the peak of the monomeric fraction divided by the total area under the curve of the chromatogram.
3. Disulfide bond reduction activity of ORP100S was quantified by assaying the reduction state of a small protein (human insulin) which in its heterodimeric form contains two intermolecular and one intramolecular disulfide, all three of which are known to be suitable thioredoxin substrates. Insulin reduction has classically been used to quantify thioredoxin activity by absorbance change following addition of NADPH and TrxR (Holmgren A., 1979, Thioredoxin catalyzes the reduction of insulin disulfides by dithiothreitol and dihydrolipoamide, J Biol Chem 254:9627-32). Such an approach is not suitable for monothiol C35S thioredoxins ORP-100 and ORP100S due to 1) the lack of cycling resulting from stoichiometric covalent linkage to the disulfide bond substrate and 2) the inability of NADPH and TrxR to reduce Cys32 of the oxidized monothiol Trx active site. Consequently, a new assay was developed based on the use of reverse-phase (RP) HPLC to monitor the rate of conversion of disulfide-bonded insulin heterodimers to monomeric forms. The two chains of human insulin have a total of six Cys residues which form three disulfide bonds in its mature structure. When ORP100S in the reduced form is incubated with dimeric insulin it reacts with these disulfide bonds to disrupt them, simultaneously forming covalent linkages to the ORP100S Cys32. This results in changes in mobility of the intact insulin heterodimer that can be detected using RP-HPLC separation, making possible quantification of the change in heterodimer peak area over time as a measure of protein disulfide reduction activity. The ORP100S samples were incubated with 10 mg/mL insulin for various time points (0-90 min) and the reactions were stopped by addition of iodacetic acid (IA) and trifluoroacetic acid (TFA). Relative activity at each time point was determined from changes in the area under the insulin heterodimer peak following separation over RP-HPLC (Agilent 1100) using an Intrada WP-RP 50×3 (Imtakt) column. Buffer A was 0.1% TFA and Buffer B was 0.1% TFA in acetonitrile. The gradient was 0 to 3% B in 5 min followed by 30 to 60% B in 45 min, then 60 to 80% B for an additional 5 min. The flow rate was 0.2 mL/min and absorbance was measured at 280 nm. In order to evaluate the change in the intact insulin molecule a time 0 baseline was first established using 1 M IA and 0.1% TFA without the addition of ORP100S. The area under the intact insulin heterodimer peak was then determined and set as equivalent to 100%. After the reaction with ORP100S the area of the intact insulin peak (corresponding to retention time) was measured. The percent reduction in intact insulin was then calculated from the decrease in the area following reduction divided by the area at time 0 multiplied by 100.

### Example 2. pH Dependence of Reducing Activity

This example illustrates that molecules of the invention having thioredoxin active sites have significantly greater activity at physiologically relevant pH than conventional thiol reducing agents due to a lower pKa value.

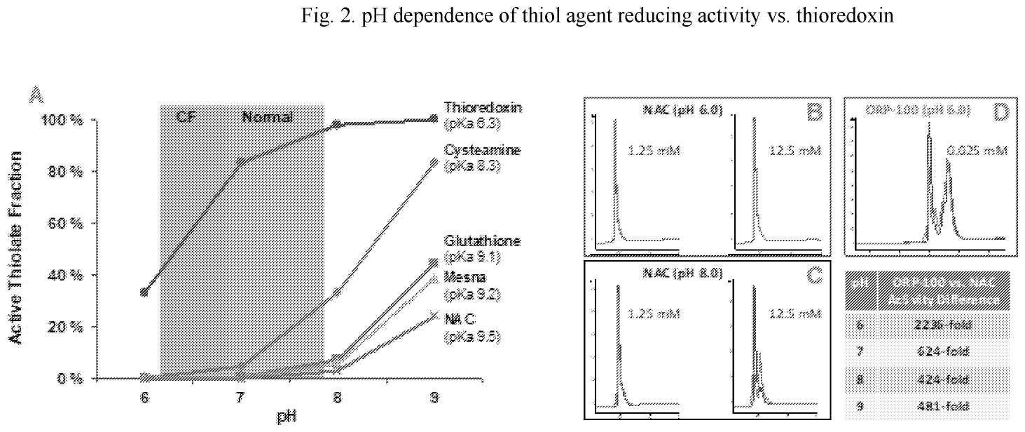

The human CF airway surface liquid is approximately 0.8 pH units more acidic than that of unaffected individuals due to the loss of bicarbonate-mediated buffering of proton secretion (Garland A L, Walton W G, Coakley R D, et al., 2013, Molecular basis for pH-dependent mucosal dehydration in cystic fibrosis airways. PNAS 110:15973-8; Shah V S, Meyerholz D K, Tang X X, et al., 2016, Airway acidification initiates host defense abnormalities in cystic fibrosis mice, Science 351:503-7). This has consequences for the clinical use of reducing agents to treat condensed, abnormal mucus. Approved and investigational thiol agents N-acetyl cysteine (NAC), glutathione (GSH), cysteamine and 2-mercaptoethane sulfonate Na (Mesna) all exhibit low levels of disulfide reducing activity at CF airway pH due to the highly basic equilibrium point (pKa) between their inactive (protonated) and active (deprotonated) forms. Cys thiol pKa values range from pH 8.5 (cysteamine) to pH 9.5 (NAC). In marked contrast to these classical thiols, the structurally-stabilized pKa of the Trx active site Cys32 is two to three logs lower (pH 6.1-6.3), allowing high activity even at acidic CF airway pH (FIG. 2). We have verified experimentally (data not shown) that ORP-100 and ORP100S share the same pKa as native Trx, demonstrating that the monocysteinic active site modifications do not interfere with the unique hydrogen bonding that stabilizes the deprotonated thiolate anion at Trx Cys32.

FIG. 2A shows the percent of thiols calculated to be in the deprotonated, active form over the pH range 6-9 for thioredoxin (and ORP-100/ORP100S) vs. four representative small-molecule thiol agents. FIG. 2B shows RP-HPLC traces for a representative insulin reduction experiment showing conversion of insulin heterodimer peak with 1.25 and 12.5 mM NAC at pH 6 (Left, top) and pH 8 (Left, bottom). Panel B, Right top, shows the trace obtained for 0.025 mM ORP-100 at pH 6. Overlapping Time 0 and 60 min traces indicate lack of reduction over the 60 min incubation time: 1.25 mM NAC is inactive for insulin reduction at pH 6 or 8, and NAC is only able to reduce insulin at 12.5 mM at pH 8, but not at pH 6, as indicated by the shaded peak area denoting conversion of insulin heterodimer to monomer at 60 min. In contrast, even at acidic pH 6 ORP-100 is able to markedly reduce the insulin heterodimer peak, and at 1/500 the concentration of NAC (FIG. 2B, right panel, top). Table: relative activities of NAC vs. ORP-100 at pH 6-9. Similar results were obtained with native Trx and ORP100S vs. NAC or GSH. These results demonstrate that monothiol active site C35S thioredoxin (ORP-100, ORP100S) retains the remarkably potent disulfide-reducing activity of thioredoxin across the full physiological pH range anticipated in the human airway, including at neutral to acidic pH levels where exogenous and endogenous (e.g GSH) small thiols are substantially inactive.

### Example 3. Correlation of ORP100S Reduction State with Activity

This example demonstrates that fully monothiol ORP100S exhibits a strong correlation between overall protein reduction state and disulfide bond reducing activity.

ORP100S was reduced with 100 mM DTT and residual reductant removed from the sample using a SEPHADEX™ G-25 column (e.g. GE Healthcare NAP-5 column) for exchange into 20 mM ammonium acetate, pH 5.5. Fully reduced ORP100S (“Red”) was mixed at different ratios with oxidized ORP100S treated with iodacetamide (IA) and exchanged into ammonium acetate buffer to remove unreacted iodoacetomide (“Ox”). The results are shown in Table 1. ORP100S solutions containing different ratios of reduced:oxidized protein were analyzed by DTNB, SEC and RP-HPLC insulin reduction assays as described in Example 1. The maximum insulin heterodimer reaction with fully reduced material was 45% for the time and conditions used. Insulin reduction values are expressed as relative reduction vs. maximum. While it is apparent from the 100% IA-treated: 0% reduced treatment that there is residual activity in the ‘fully-oxidized’ sample the results nonetheless confirm that monothiol Trx ORP100S exhibits very good linear correlation between overall reduction state and disulfide bond reducing activity, unlike native Trx with five reducible Cys or ORP-100 with four (not shown). For example, with four total Cys ORP-100 can be as much as 75% reduced and still have 0% insulin reduction activity when the active site Cys32 is fully oxidized by dimerization. We have verified that oxidation proceeds primarily via intermolecular disulfide formation to create ORP100S homodimers (and in the case of Trx or ORP-100, higher-order multimers as well).

TABLE 1

| ORP100S | | DTNB | SEC | Relative % insulin |
|---|---|---|---|---|
| Ox | Red | % reduced | % monomer | reduction |
| 100 | 0 | 22 | 22 | 8 |
| 75 | 25 | 49 | 36 | 36 |
| 50 | 50 | 55 | 52 | 51 |
| 25 | 75 | 73 | 70 | 72 |
| 0 | 100 | 86 | 89 | 100 |

### Example 4. Stability in Solution vs. Lyophilized

This example demonstrates that monothiol active site thioredoxins ORP-100 and ORP100S produced at laboratory scale using the initial manufacturing process described in Example 1 are significantly more stable when lyophilized as pure protein from a volatile solvent than when stored as solutions of the compounds as measured by free SH groups and percent monomers. The stability was comparable to that obtained using complex sucrose and EDTA formulations that previously were the only formulations able to maintain thioredoxin in the reduced form during prolonged storage.

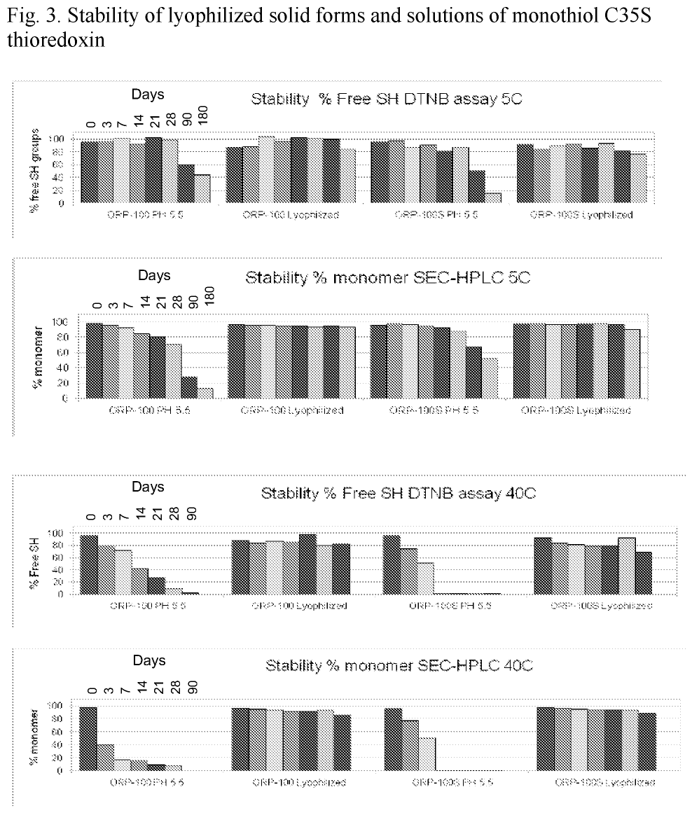

Prior work, e.g. WO 2006/090127, teaches compositions for maintenance of thioredoxin in the reduced state. Significant experimentation was required to derive compositions able to keep thioredoxin reduced during storage, and these compositions were complex and required saccharide derivatives and EDTA as excipients. Our discovery, that elimination of all excipients by means of solubilizing reduced thioredoxin in aqueous solutions incorporating volatile solvents that sublimate away during lyophilization could result in comparable stability, was therefore unexpected. The results of a stability analysis using such a formulation strategy are shown in FIG. 3. ORP-100 and ORP100S proteins were reduced with 100 mM DTT and exchanged over a NAP-5 column into volatile 20 mM ammonium acetate buffer at pH 5.5 to remove residual reductant. Half of the material was frozen at −80° C. then lyophilized while the remainder was kept in solution at either 5° C. or 40° C. for various time points (“PH5.5”). Lyophilized protein was reconstituted back into 20 mM ammonium acetate pH 5.5 immediately prior to evaluation at each time point (“Lyophilized”). Stability was assessed by measuring both free SH groups (DTNB chromogenic assay) and the percent monomers (SEC-HPLC). As shown in FIG. 3, excellent storage stability was obtained in the lyophilized form even after six months under accelerated stability conditions at 40° C. While largely similar to ORP-100, ORP100S exhibited slightly better monomer stability by SEC-HPLC, particularly during the first week of storage in liquid formulation. Based on our prior results the higher DTNB for ORP-100 vs. percent monomer reflects reduction of non-active site Cys that do not contribute to activity and which are not present in ORP100S (see Example 3).

For FIG. 3: PH5.5: ORP-100 or ORP100S protein maintained in liquid formulation of 20 mM ammonium acetate, pH 5.5 for 0, 3, 7, 14, 21, 28, 90, or 180 days. Lyophilized: ORP-100 or ORP100S protein stored in the lyophilized form at 5° C. or 40° C. for the 0, 3, 7, 14, 21, 28, 90, or 180 days and assayed following reconstitution in 20 mM ammonium acetate, pH 5.5. Percent free sulfhydryl (reduction state) at 5° C. Second panel: Percent monomer determined by SEC assay at 5° C. Third panel: Percent free sulfhydryl at 40° C. Fourth panel: Percent monomer determined by SEC assay at 40° C.

### Example 5: Large Scale Manufacture and Removal of High UV Absorbance Thioredoxin Protein Fraction

This example demonstrates the production of a thioredoxin protein composition of the invention from which a thioredoxin protein fraction having UV absorbance above 400 nm was removed and describes the stability and water absorption characteristics of the resulting composition.

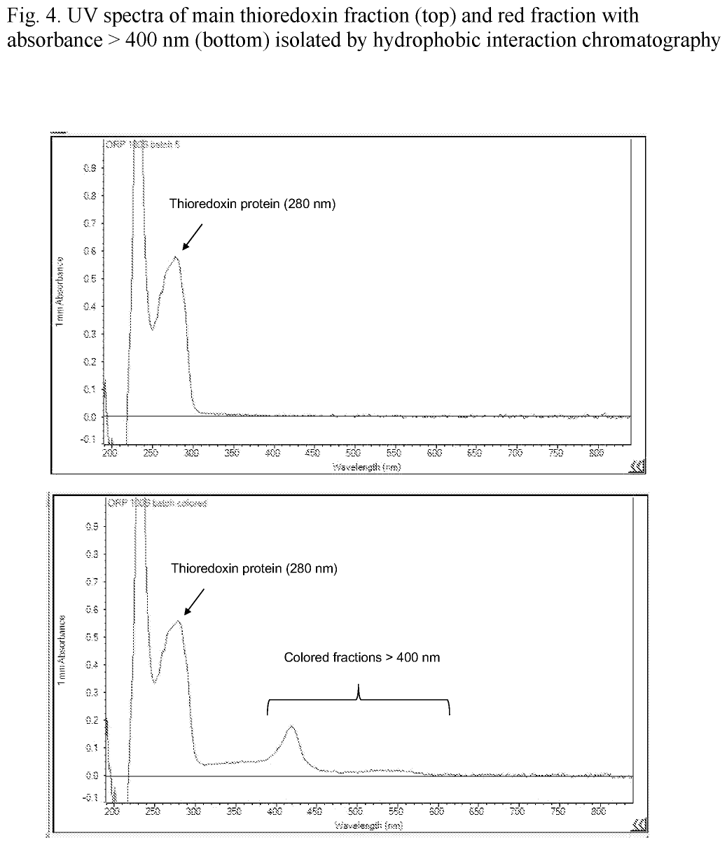

A composition comprising ORP100S was prepared by culturing rhaB-E. coli recombinantly engineered to express the protein in a 150 L fermentor and inducing expression with rhamnose addition. The resulting fermentation broth was harvested 48 hr post-induction and homogenized to lyse the cells, following which the lysate was frozen for further processing. The final titer of ORP100S in the 105 L fermentation broth was 16 g/L. The frozen lysate was thawed and clarified by standard techniques. The protein composition in the clarified lysate was subjected to a first anion-exchange chromatography step (Capto Q resin 15 L−5L x 3, cat 117531604, GE Healthcare) in bind and elute mode, and a second anion-exchange chromatography step (Sartobind STIC PA chromatography —Sartorius) in flow through mode for endotoxin removal. The resulting protein composition was subjected to hydrophobic interaction chromatography (Capto Phenyl ImpRes 10L−5L×2, Cat 17548404, GE Healthcare) in bind and elute mode. This HIC-purified composition included a main thioredoxin protein fraction having a single UV absorbance peak at about 280 nm, and a second thioredoxin protein fraction (corresponding to ca. 10% of the thioredoxin amount) having in addition a prominent UV absorbance peak at about 423 nm as well as minor peaks in the 500-600 nm range (FIG. 4). The minor fraction was colored yellowish-pink (“red fraction”), while the main fraction was substantially clear. All purification steps were conducted in the presence of DTT to maintain complete reduction.

Both the main fraction and the red fraction were individually exchanged into ammonium acetate buffer pH 5.5 by ultrafiltration/diafiltration, and frozen at −80° C. in 500 ml bottles. Complete removal of DTT was verified by HPLC-MS. The composition was dried by lyophilization as follows. The frozen product was thawed by transfer from −80° C. to a 2-8° C. refrigerator for 60 hours. The thawed product was filtered using a 0.2 uM PES filter, 500 mL sterile filter/bottle combination. The filtered product was transferred to Gore LyoGuard lyophilization trays (1.5L product/tray, equivalent to 3×500 mL product bottles), and the filled trays were transferred to the lyophilizer shelf with a temperature probe placed on top of the tray. The lyophilization cycle was started after purging with nitrogen gas.

Five batches of product were lyophilized according to the following lyophilization cycle program.

TABLE 2
Lyophilization Cycle Program

| | | | | | Hold/Ramp | |
|---|---|---|---|---|---|---|
| Cycle | Hours | Temp (C) | Ramp/Hold | Ramp rate | Time (hr) | Total Hours |
| Freezing | 0 | 20 | Ramp | 0.5 C/min | 1.67 | 1.67 |
| | 1.67 | −30 | Hold | | 10 | 10.67 |
| | 11.67 | −30 | Ramp | 0.5 C/min | 0.83 | 11.50 |
| Primary | 12.50 | −5 | Hold | | 50 | 61.50 |
| Drying | 62.5 | −5 | Ramp | 0.5 C/min | 1.00 | 62.50 |
| Secondary | 63.50 | 25 | Hold | | 10 | 72.50 |
| Drying | 73.50 | 25 | Ramp | 0.5 C/min | 0.33 | 72.83 |
| | 73.83 | 35 | Hold | | 10 | 83.83 |
| | 83.83 | 35 | Ramp | 0.5 C/min | 0.167 | 84.00 |
| | *84.16 | 40 | Hold | | 10 | 94.00 |

| | | | | |
|---|---|---|---|---|
| *94.16 | 40 | End cycle | Total time | 94.00 |

After lyophilization, the trays were purged with nitrogen, and the lyophilization cakes were broken into powder that was packaged in sterile storage bottles for storage at −20° C. (final recovery 88.7%). The product was then assayed for moisture content which in the main fraction ranged from 0.81 wt % to 2.18 wt % across the five batches. In contrast, the red thioredoxin fraction could only be dried to a minimum water content of about 6.0 wt %.

Characterization: After drying and reconstitution in saline, the main fraction showed comparable disulfide reduction activity by DTNB and RP-HPLC to thioredoxin compositions purified without removing the red fraction in a HIC purification step. However, the main fraction compositions lacking the red fraction with UV absorbance >400 nm demonstrated considerably greater stability when reconstituted into saline and maintained at room temperature as compared to thioredoxin compositions purified by methods insufficient to remove the colored fraction. The stability of ORP100S without the red fraction was evaluated at room temperature (25° C.) by SEC analysis as described in the preceding Examples. Protein solutions were prepared in PBS (saline) at concentrations of 70, 80, 90, 100 and 110 mg/ml corresponding to 6.0, 6.8, 7.7, 8.5 and 9.4 mM. These were incubated at room temperature for 0, 20, 44, 68, 140, 188 and 232 hours. At each time point tubes were centrifuged at 1000 RFC to check for precipitates and percent monomeric ORP100S determined by SEC. Additionally, at the 68 hr time point DTNB assays were performed to evaluate the degree of reduction. Virtually no change was observed in percent monomer across all concentration levels, which decreased only 2% from time 0 to 232 hours (see table below). By comparison, an ORP100S composition without removal of the red fraction reconstituted in PBS (saline) at a comparable concentration of 5 mM (59 mg/ml) had a starting percent monomer fraction of 95% at time 0 which decreased to 55% at 72 hr and 26% at 168 hr when incubated at 25° C.

TABLE 3

| | % | | | |
|---|---|---|---|---|
| ORP100S | monomer | % monomer | % reduced | Precipitate |
| concentration | (Time 0) | (232 hrs) | (68 hrs) | (all time points) |
| 70 mg/mL | 93 | 93 | 109 | None |
| 80 mg/mL | 94 | 93 | 87 | None |
| 90 mg/mL | 94 | 92 | 91 | None |
| 100 mg/mL | 95 | 93 | 91 | None |
| 110 mg/mL | 95 | 92 | 102 | None |

Overall, the material purified as described with removal of the red fraction had greater purity and was more uniform in appearance, with extremely low endotoxin levels.

TABLE 4

| | |
|---|---|
| Concentration: | 68.4 mg/ml |
| Appearance: | |
| Opacity: | Clear |
| Visible particulates: | None |
| Color: | Colorless |
| Purity: | |
| SDS-PAGE (non-reducing): | 97.9% (2.1% HMW) |
| SDS-PAGE (reducing): | 100% |
| SEC-UPLC: | 99.6% (0.4% HMW) |
| Safety: | |
| Endotoxin level (Endosafe PTS): | 0.002 EU/mg |

### Example 6. ORP100S Mucus Rheology and Mucin Molecular Weight Reduction

This example demonstrates the efficacy of monocysteinic human thioredoxin-1 ORP100S to reduce the viscoelastic properties of human CF mucus as well as the molecular weight (MW) of mucin glycoproteins. The ability of ORP100S to reduce viscous and elastic moduli of 4% CF mucus cultured in vitro from primary human bronchial epithelia (HBE) was evaluated, as well as the effect of ORP100S treatment on mucin polymer size using gel permeation chromatography (GPC)/multi-angle light scattering (MALLS). Together these results demonstrate a potent ability of ORP100S to normalize CF mucus and sputum viscoelasticity, as well as mucus transportability, and suggest that ORP100S may be a potential CF treatment optimized for activity across a broad airway pH microenvironment.

### Methods:

Mucus Preparation: Aseptic mucus was harvested from over 100 individual HBE cultures from 20 different CF donors and prepared to four weight-percent (4%) solids, a concentration that typifies chronic obstructive pulmonary disease (COPD) and mild CF (Hill, D. B. et al., 2014, A biophysical basis for mucus solids concentration as a candidate biomarker for airways disease, PloS one 9, e87681; Anderson, W. H. et al., 2015, The relationship of mucus concentration (hydration) to mucus osmotic pressure and transport in chronic bronchitis., Am J Resp Crit Care Med 192: 182-90).
Rheology: HBE cell culture mucus was treated with DTT (1 mM) and several concentrations of ORP100S (0.01, 0.1, and 1.0 mM) for 1 hr at 37° C. following previously established methods (Hill, D. B., and Button, B., 2012, Establishment of respiratory air-liquid interface cultures and their use in studying mucin production, secretion, and function. In Mucins: Methods and Protocols, D. J. Thornton, ed., pp. 245-58; Youngren-Ortiz, S. et al., 2017, Development of optimized inhalable Gemcitabine-loaded gelatin nanocarriers for lung cancer, J Aerosol Med Pulm Drug Delivery 30:299-321; Seagrave, J., et al., 2012, Effects of guaifenesin, N-acetylcysteine, and ambroxol on MUC5AC and mucociliary transport in primary differentiated human tracheal-bronchial cells, Respir Res 13: 98). Concentration and time course assays were performed using a TA Instruments DHR3 rheometer to assess the bulk, macroscopic biophysical effects of test article and controls on HBE mucus properties. Briefly, the linear regime of a 1 Hz amplitude (stress) sweep was identified for each treatment condition. The frequency of 1 Hz was selected because it is between the frequencies associated with tidal breathing (˜0.25 Hz) and mucociliary clearance (10-15 Hz), and has been shown to correlate to mucociliary clearance (Tomkiewicz, R. et al., 1994, Mucolytic treatment with N-acetylcysteine L-lysinate metered dose inhaler in dogs: airway epithelial function changes. Eur Resp J 7: 81-87). Creep recovery experiments were performed in which a known stress (between 0.05 and ˜100 Pa) was applied to treated or control mucus for 10 seconds, and the rheological recovery of the fluid was recorded for an additional 50 seconds. In successive runs, the applied stress was increased in a logarithmic fashion until the yield stress of the fluid was reached (i.e., the stress at which the viscosity of the fluid suddenly and dramatically decreased). From the measured parameters the viscosity and elasticity of the fluid were determined as a function of applied stress. Frequency sweeps were performed at both constant stress and strains and used to determine the baseline physical properties of mucus and its elastic and viscous components (G′ and G″, respectively).
Mucin molecular weight determination: Molecular weight reduction following test article treatment was determined using a combination of gel-permeation chromatography with multi-angle laser light scattering on a Wyatt Heleos MALLS system. MALLS is a rapid and accurate means of determining molecular size and mass of high MW biomolecules in a non-destructive manner without requiring the use of relative standards. Briefly, 4% HBE treated samples were diluted 100-fold in 0.9% NaCl with 10 mM EDTA and 0.01% sodium azide. 0.2 mL of diluted sample was eluted through a Sepharose CL2B column to separate high MW mucins from other mucus proteins, and the mucin fraction was introduced into the MALLS system. Mucin MW was determined by fitting a Berry model to light scattering from 11 different angles using Wyatt Astra software.

### Results

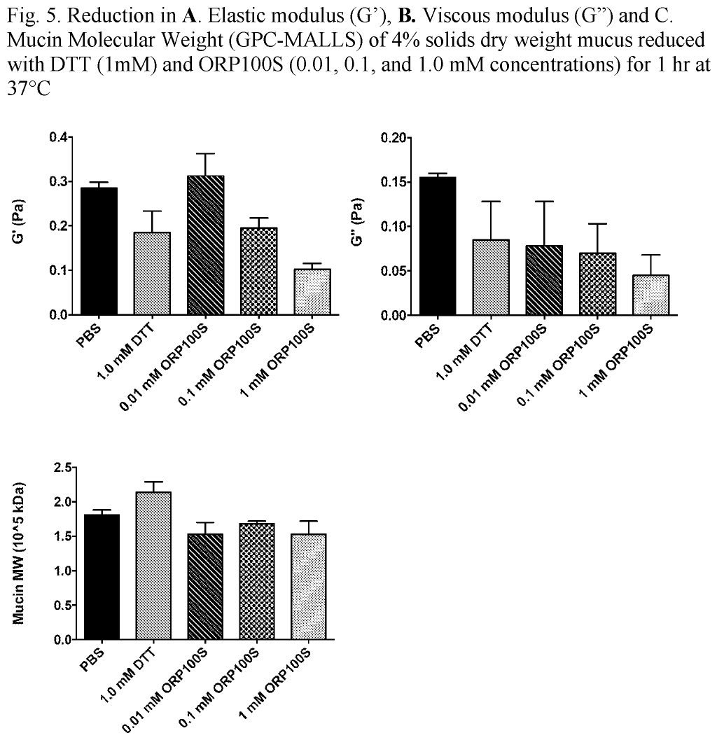

Viscoelasticity: ORP100S demonstrated a concentration-dependent ability to decrease both the elastic (storage, G′) and viscous (loss, G″) moduli of 4% HBE mucus (FIG. 5, top). At the lowest tested ORP100S concentration (10 μM), significant reduction in G′ was not apparent, while a nearly 2-fold reduction in G″ was observed. At 100 μM ORP100S G′ was decreased from a baseline value of 0.28 Pa to 0.19 Pa, with G″ reduced by a similar amount as observed with 10 μM. The degree of rheological reduction achieved by ORP100S at this concentration was similar to that obtained with 1 mM DTT, a strong dithiol reductant with potent mucolytic properties. Strikingly, 1 mM ORP100S showed markedly greater rheological reductive properties than DTT, reducing both G′ and G″ by nearly a factor of 3.

FIGS. 5, A and B (top): Reduction in Elastic (G′) and Viscous (G″) moduli of 4% solids dry weight mucus reduced with DTT (1 mM) and ORP100S (0.01, 0.1, and 1.0 mM concentrations) for 1 hr at 37° C. Results show that 0.1 mM ORP100S reduces G′ as effectively as a ten-fold higher concentration of DTT. All data were collected by examining frequency sweeps of mucus performed in the linear regime and analyzed at 1 rad/s on a TA DHR3 rheometer.

Mucin size: Unlike 1 mM DTT, which demonstrated a modest increase in the molecular weight of mucins (from 180 MDa to 210 MDa), all three concentrations of ORP100S reduced the molecular weight of mucins equivalently to ca. 150 MDa (FIG. 5, C bottom). All compounds reduced the concentration of mucins present in the refractometry system (data not shown). The mild increase in the average molecular weight of mucins with 1 mM DTT is a possible signature of the compound opening reactive cysteine residues, which could allow mucin macromolecules to interact with themselves as well as with other mucus proteins. Monothiol reductants like ORP100S that cap free Cys thiols would not be expected to react in this manner, nor would higher concentrations of DTT that completely reduce mucin macromolecules to monomers.

FIG. 5C. Mucin molecular weight reduction (GPC-MALLS) of 4% solids dry weight mucus reduced with DTT (1 mM) and ORP100S (0.01, 0.1, and 1.0 mM concentrations) for 1 hr at 37° C.

### Conclusions:

ORP100S demonstrated a markedly greater capacity to reduce the rheology of abnormally viscoelastic CF mucus, mole per mole, than DTT. Importantly, the potent viscoelasticity modulating effect of ORP100S did not result in complete reduction of mucins and polymer disassembly, suggestive of a degree of enzymatic selectivity for intramolecular mucin disulfides that increase polymer density, over intermolecular disulfides that link mucin monomers into a functional gel. This mucus normalization as opposed to mucolysis is consistent with the expected behavior of a native airway mucus disulfide homeostatic mechanism based on a redox cycle of thioredoxin, glutathione and glutaredoxin, of which all three components are present in airway surface liquid in vivo (Du, Y., Zhang, H., Lu, J. & Holmgren, A., 2012, Glutathione and glutaredoxin act as a backup of human Thioredoxin Reductase 1 to reduce Thioredoxin 1 preventing cell death by aurothioglucose, J Biol Chem 287, 38210-19; Bartlett, J. A. et al., 2013, Protein composition of bronchoalveolar lavage fluid and airway surface liquid from newborn pigs, Am J Physiol—Lung Cell Mol Physiol 305:L256-66).

### Example 7. CF Mucus and Sputum Transportability Following Treatment with ORP100S

This example demonstrates that ORP100S increases CF mucus and sputum transportability in vitro in cultured primary human bronchial epithelial cells, and in situ on excised adult rat tracheae.

### Methods

### Primary Human Bronchial Epithelial Studies

Primary human bronchial epithelial (HBE) cells were derived from lung explants from healthy failed donors and CF patients homozygous for F508del CFTR. Cells were expanded and grown to confluency, seeded onto 6.5 mm diameter permeable supports (0.5×106 cells/filter; Corning) coated with NIH 3T3 fibroblast unconditioned media, and grown in differentiating media for at least 6-8 weeks until terminally differentiated (Birket S E, Chu K K, Houser G H, Liu L, Fernandez C M, Solomon G M, Lin V, Shastry S, Mazur M, Sloane P, et al., 2016, Combination therapy with Cystic Fibrosis Transmembrane Conductance Regulator modulators augment the airway functional microanatomy, Am J Physiol Lung Cell Mol Physiol. ajplung 00395; Birket S E, Chu K K, Liu L, Houser G H, Diephuis B J, Wilsterman E J, Dierksen G, Mazur M, Shastry S, Li Y, et al., 2014, A functional anatomic defect of the cystic fibrosis airway, Am J Respir Crit Care Med. 190(4):421-32). Cells were washed with PBS and allowed to grow for 48 hrs in order to re-establish a fresh mucus layer before apical treatment (to mimic aerosol deposition) with ORP100S (1-3 mM), vehicle control (PBS; −MG++, −Ca++), or positive control DTT (1.6 mM; Sigma-Aldrich, St. Louis, Mo.). Micro optical coherence tomography (μOCT) images were obtained at baseline and 3 hrs post treatment for 3-4 monolayers per condition at 4 regions of interest per monolayer. Only first or second passage cells were used.

### Effect of ORP 100S on Sputum Transport Ex Vivo

To determine the effect of ORP100S on CF sputum transportability, sputum specimens spontaneously expectorated from 4 CF patients hospitalized for pulmonary exacerbation were collected and stored at 4° C. before being divided into 200 μL aliquots and treated with ORP100S (3 mM), PBS, DTT (1.6 mM), or DNase (10 or 25 μg/ml) on the day of or day following collection. Upon treatment, sputum aliquots were placed in a 37° C. water bath for 2 hrs and then applied (3 μl per sample) for μOCT imaging to the distal end of trachea excised from adult non-CF rats. Trachea were washed 2× with 500 μl of PBS before sputum addition. Sample conditions were applied in triplicate in random order at distinct anatomic locations—2 washes using 500 μl of PBS were performed between each sample addition—and at least 3 images were collected from each region of interest. Confirmation of trachea viability was obtained by imaging with PBS upon completion of the experiment.

### μOCT Imaging

One-micron resolution spectral domain μOCT was used to obtain measurements of mucociliary transport (MCT) rate and ciliary beat frequency (CBF) in HBE monolayers and of MCT rate of sputum ex vivo. This first-in-kind, high-speed (40 frames per second, 512 lines per frame) microscopic reflectance imaging modality enables simultaneous anatomic imaging in cell cultures and intact tissues that readily distinguishes the properties of CF compared to normal epithelia (Liu L, Chu K K, Houser G H, Diephuis B J, Li Y, Wilsterman E J, Shastry S, Dierksen G, Birket S E, Mazur M, et al., 2013, Method for quantitative study of airway functional microanatomy using micro-optical coherence tomography, PLoS One 8(1):e54473; Tuggle K L, Birket S E, Cui X, Hong J, Warren J, Reid L, Chambers A, Ji D, Gamber K, Chu K K, et al., 2014, Characterization of defects in ion transport and tissue development in Cystic Fibrosis Transmembrane Conductance Regulator (CFTR)-knockout rats. PLoS One 9(3):e91253). In addition to MCT rate and CBF, μOCT also has the capability to assess physical characteristics of the airway surface liquid. Images were captured, and MCT rate and CBF calculated, as described in the preceding references.

### Statistical Analysis

Inferential statistics (mean, SD, SE) were computed using ANOVA, and Tukey's post-hoc test was used for multiple comparisons where appropriate. Statistics are presented as mean±SE, with P values <0.05 considered as significant. All statistical analyses were performed using GraphPad Prism version 7.0a (La Jolla, Calif.).

### Results

### ORP100S Augments MCT Rate in Non-CF and CF Primary HBE Cells

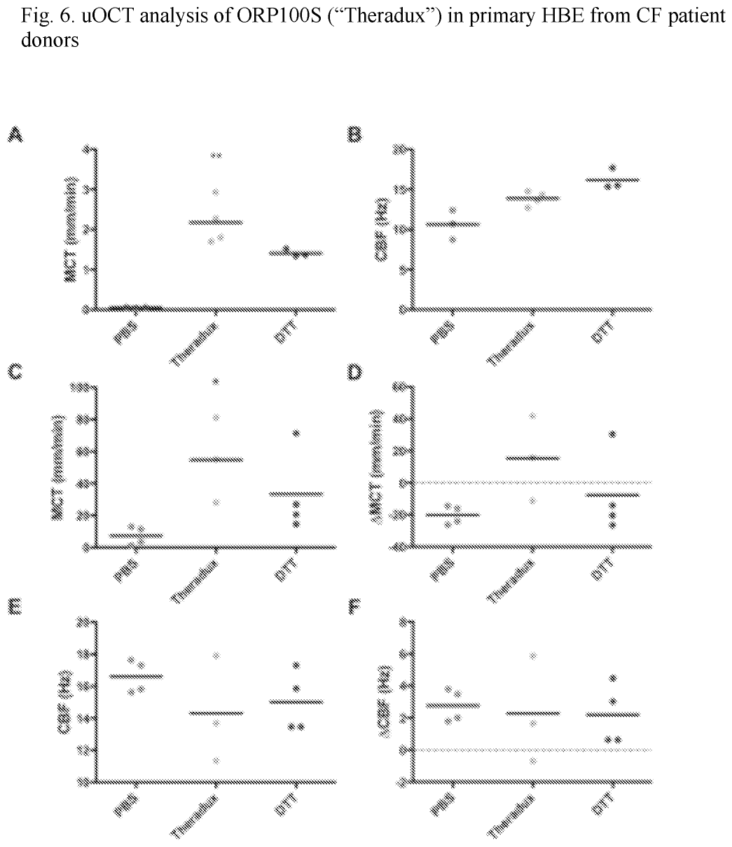

To determine whether ORP100S alters mucus transport, we assessed its effect on MCT rate and CBF in primary HBE cells derived from healthy non-CF donors and CF donors homozygous for F508del. For these studies, we used μOCT imaging, which enables measurement of these and other parameters of the airway functional microanatomy without the use of exogenous particles or dyes. Results demonstrate that ORP100S (“Theradux”)-treated (1-3 mM) non-CF cells exhibited a significantly higher MCT rate (2.18±0.3 mm/min, P<0.01) vs. PBS (0.05±0.007 mm/min) at 3 hrs post-treatment that exceeded the effect of DTT (1.6 mM; 1.41±0.1 mm/min) (FIG. 6A). This effect was recapitulated in CF cells, which displayed a significantly elevated MCT rate at 3 hrs with ORP100S (54.73±15.3 mm/min, P<0.05) vs. PBS (7.30±2.9 mm/min) or DTT (33.33±12.9 mm/min) that increased from baseline (15.4±15.3 mm/min) (FIGS. 6C and D). ORP100S elicited no meaningful differences in CBF in either non-CF or CF cells (FIGS. 6B, E, and F), in support of its disulfide reducing properties as a mechanism driving improvements in MCT. FIG. 6. μOCT analysis of ORP100S (Theradux) in primary HBE cells (non-CF and CF). (A) Raw mucociliary transport (MCT) rate and (B) ciliary beat frequency (CBF) at 3 hrs post treatment for non-CF cells. For CF cells, (C) raw MCT rate at 3 hrs and (D) change in MCT rate vs. baseline at 3 hrs post treatment. (E) Raw CBF and (F) change in CBF vs. baseline also were measured. N=3-4 monolayers per condition across 1 non-CF and 1 CF donor. Each data point represents mean treatment effect per monolayer. N=3-4 monolayers per condition. * P<0.05 ** P<0.01

### ORP100S Improves Mucus Clearance in Intact Trachea

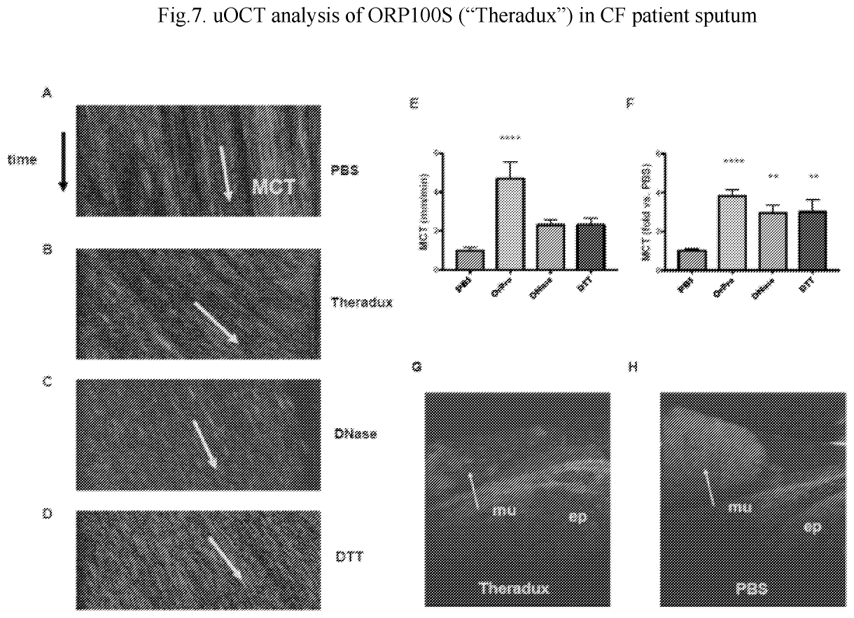

The effect of ORP100S on mucus clearance using intact rat trachea, which include the complexities of the airway surface such as gland expression of a fully differentiated mucosal surface, was further evaluated. Spontaneously expectorated sputum was collected from four CF patients with genotypes F508del/F508del, F508del/S589N (N=2), and F508de1/1973_1985del13InsAGAAA (mean age=31 yrs; mean FEV1=1.62 L) and treated with ORP100S (3 mM), PBS, DTT (1.6 mM), or DNase (10 or 25 μg/ml) for 2 hrs. Sputum was then added to the surface of living wild type rat trachea and imaged using μOCT under physiologic conditions. FIG. 7A-D, display representative re-sliced μOCT images depicting mucus transport of each treatment condition. MCT was measured by projecting a cross-sectional line through the mucus through time, with the slope of the particle trajectories indicating velocity. As summarized in FIGS. 7E and F, ORP100S-treated cells indicated a higher MCT rate (4.68±0.9) mm/min, P<0.0001) vs. PBS (0.97±0.17 mm/min) that significantly exceeded the effect of standard of care DNase (2.30±0.28 mm/min) and DTT (2.31±0.34 mm/min). The change in MCT rate normalized to the effect of PBS was 3.80±0.35 mm/min (P<0.0001), again surpassing that observed with DNase (2.95±0.40 mm/min, P<0.01) or DTT (3.01±0.61 mm/min, P<0.01). As seen in representative μOCT images of ORP100S vs. PBS-treated sputum (FIGS. 7G and H), ORP100S was effective in decreasing sputum density, in line with previously observed viscoelasticity data.

FIG. 7. μOCT analysis of ORP100S (“Theradux”) in CF sputum. (A-D) Representative re-sliced μOCT images of each treatment condition depicting mucus transport. MCT was measured by projecting a cross-sectional line through the mucus through time. The slope of the particle trajectories indicated velocity. (E) Raw mucociliary transport (MCT) rate and (F) change in MCT rate normalized to PBS across sample samples from N=4 CF donors. Representative images depicting (G) ORP100S effect on mucus density vs. (H) PBS (mucus (mu), epithelium (ep). Mean +/−SEM, ** P<0.01, **** P<0.0001.

### Conclusions

ORP100S-treated primary non-CF and CF HBE cells exhibited increased MCT that exceeded the effect of DTT. ORP100S augmentation of mucus transportability vs. positive controls also was observed in expectorated CF sputum, with accompanying decrease in sputum density. Together, these results indicate that reducing disulfide bonds by ORP100S increases mucus transportability, and suggest that ORP100S may be a potential CF treatment optimized for activity across a broad airway pH microenvironment.

### Example 8. ORP100S in Reduced Form is Non-Inflammatory In Vitro

In vitro studies in normal and CF HBE cultured at an air-liquid interface (HBE-ALI) were conducted to evaluate the potential for monothiol thioredoxin to induce inflammatory cytokine release following microspray application to cell monolayers.

Nasal airway epithelial cells from normal healthy volunteers or CF subjects were cultured in serum-free media at an air-liquid interface with mucociliary differentiation, based on methods adapted from the approach of Schlegel and colleagues (Suprynowicz, F. A., et al., Proc Natl Acad Sci USA, 2012, 109(49): p. 20035-40; Becker, M. N., et al., Am J Respir Crit Care Med, 2004, 169(5): p. 645-53). A number of unique CF and healthy control specimens were collected, expanded, and cryopreserved using this technique. 30 wells were cultured to differentiation at ALI for over 30 days from each of three unique normal or CF donors (homozygous for F508del). Triplicate cultures were exposed at the apical surface to PBS, native Trx, or ORP-100 and both apical and basolateral media samples were collected at 4 and 24 hours after challenge. Apical collection occurred by placing 200 uL of sterile PBS on the apical surface and recovering this after 15 min incubation. ELISA for IL-6 was performed for each sample in duplicate and others were measured using a multiplexed assay. Media was centrifuged to remove debris and stored at −80 C until ELISA analysis.

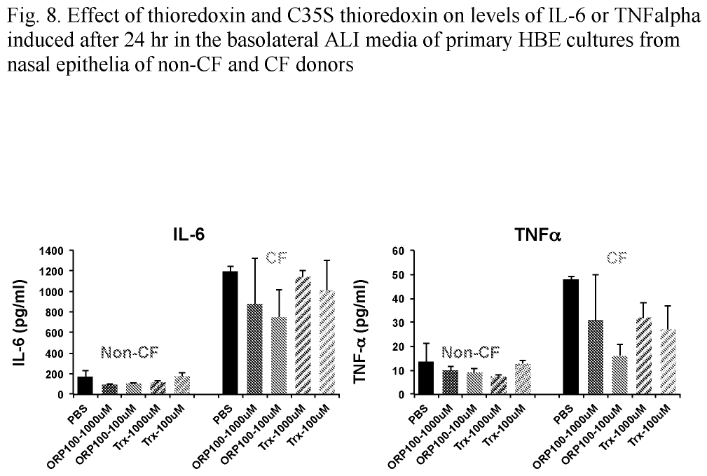

Representative data for changes in proinflammatory cytokines IL-6 (left) and TNF-alpha (right) with addition of saline (PBS) in the presence or absence of ORP-100 or thioredoxin are shown in FIG. 8 for HBE-ALI derived from healthy and CF donors. These data demonstrate that ORP-100 in the reduced form is non-inflammatory, and exhibits a significant anti-inflammatory effect at concentrations above 100 uM for drug formulated in normal (isotonic) PBS, vs. application of PBS vehicle alone, as it was observed that in CF HBE-ALI (but not those derived from healthy donors) application of isotonic or hypertonic saline was sufficient to induce both TNF-alpha and IL-6. Reduced but not oxidized ORP-100 abrogated the inflammatory effect of saline. These results were recapitulated in vivo in acute intratracheal instillation studies in normal rats.

FIG. 8: Levels of IL-6 or TNFα induced after 24 hr in the basolateral ALI media of primary HBE cultures from nasal epithelia of non-CF (left series of bars) and CF donors (right series of bars). Delivery: apical-surface bolus application for 15 min of 200 uL volumes of control or test article solutions. PBS: 0.9% phosphate-buffered saline negative vehicle control (black bars); ORP100-1000: 1 mM ORP-100 in PBS (solid dark bars); ORP100-1000: 100 uM ORP-100 in PBS (solid light bars); Trx-1000: 1 mM native thioredoxin-1 in PBS (dark hatched bars); Trx-100: 100 uM native thioredoxin-1 in PBS (light hatched bars). All concentrations reflect volumes delivered to the HBE apical surface.

### Example 9: ORP100S in the Reduced Form is Anti-Inflammatory In Vivo

This example evaluated and compared the in vivo inflammatory potential among three forms of C35S monothiol Trx (oxidized ORP-100, reduced ORP-100, and reduced ORP100S) formulated in the sucrose/EDTA formulation composition described in PCT WO 2006/090127 and delivered to normal rats as nebulized aerosols at doses of 4 and 40 mg/kg.

All three forms of ORP-100 were lyophilized following reduction with DTT and formulated in sucrose buffer (9% sucrose 1.17 mM EDTA pH 5.2). All test articles were evaluated at low (10 mg/kg) and high (40 mg/kg) target delivered doses. The test articles were administered to Charles River Sprague-Dawley rats via inhalation as an aerosol. The aerosol of each of the test articles was produced by a commercially available vibrating mesh nebulizer whose output was attached to a nose-only exposure system.

Target doses were achieved via modulation of the aerosol exposure time while maintaining the aerosol concentration constant. Animals in Low Dose groups received a single dose of test article during a 20 minute exposure to achieve a target delivered dose of 10 mg/kg and deposited dose of 1 mg/kg. Animals in High Dose groups received a single dose of test article during a 75 minute exposure to achieve a target delivered dose of 40 mg/kg and target deposited dose of 4 mg/kg.

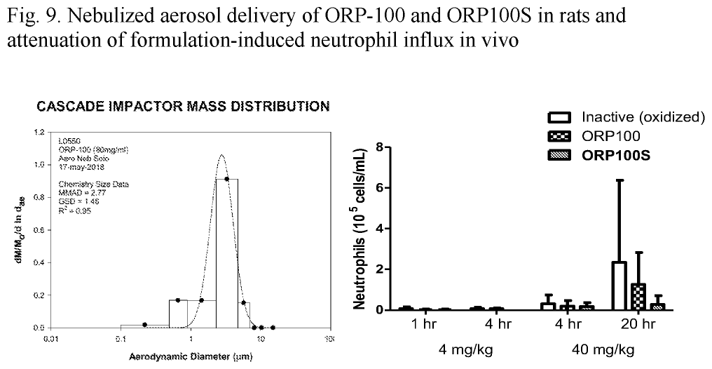

The concentration for total ORP-100 in the aerosol was determined by BCA analysis method and ranged from 601.1-868.0 μg/L for Low Dose groups and 708.7-798.0 μg/L for High Dose groups. Aerosol particle sizes were under 3.0 micron MMAD as determined by cascade impactor analysis (FIG. 9, left). Achieved deposited doses were 0.9, 1.2, and 1.2 mg/kg for Low Dose oxidized ORP-100 and reduced ORP-100 and ORP100S, respectively; and 3.8, 3.8, and 4.2 mg/kg, respectively, for High Dose groups, assuming a 10% deposition fraction. For all dose groups, actual deposited doses were within 25% of the target.

Animals in each study group were evaluated for potential toxicity by measuring various parameters including: clinical signs, body weight, clinical pathology (BALF cell counts, LDH and albumin measurements), lung tissue cytokines, lung weights, gross pathology, and microscopic pathology of respiratory target tissues. Additionally, three animals in each Low Dose group had lung tissues collected for immunohistochemistry (Alizèe Pathology, Thurmont, M D). Three (3) animals in each Low Dose group were to be euthanized at 1 hour and 4 hours post exposure for blood and tissue collections. Four (4) animals in each High Dose group were euthanized at 4 and 20 hours post exposure for blood and tissue collections.

TABLE 5
Exposure and Sacrifice Study Design

| | | | Target | | |
|---|---|---|---|---|---|
| | | | Deposited | | |
| | | | Dose | Exposure | |
| Group | | | (mg/kg), | Duration | Necropsy Time |
| ID | Exposure | N | Route | (min) | Points |
| 1 | Oxidized ORP- | 6 | 1, INH | 20 | (n = 3) at 1 and 4 |
| | 100 Low Dose | | | | hours post exposure |
| 2 | Oxidized ORP- | 8 | 4, INH | 75 | (n = 4) at 4 and 20 |
| | 100 High Dose | | | | hours post exposure |
| 3 | ORP-100 Low | 6 | 1, INH | 20 | (n = 3) at 1 and 4 |
| | Dose | | | | hours post exposure |
| 4 | ORP-100 High | 8 | 4, INH | 75 | (n = 4) at 4 and 20 |
| | Dose | | | | hours post exposure |
| 5 | ORP100S Low | 6 | 1, INH | 20 | (n = 3) at 1 and 4 |
| | Dose | | | | hours post exposure |

Differences in BALF LDH, albumin, cell counts and differential counts were unremarkable when the reduced ORP-100 and ORP100S were compared to the oxidized ORP-100 control groups, as were measurements of various lung tissue cytokines. Though slightly higher in Low Dose groups, LDH levels were variable among test article and dose groups with no apparent trend. Albumin levels were highest at the 1 hour time point for all dose groups but were similar among all test article and dose groups at 4 and 20 hours post exposure.

BALF total cells were generally similar among the three test articles regardless of dose level or evaluation time. Macrophages were the most prevalent cell type observed in BALF and showed no apparent trend among the three test articles. Most groups had low lymphocyte, neutrophil and eosinophil counts at all time points; however, slightly higher cell counts were observed in all High Dose groups sacrificed at 20 hours. BALF %-macrophages were similar among all Low Dose 1 and 4 hour time points as well as in High Dose 4 hour animals. BALF %-macrophages were slightly decreased at the 20 hour time point compared to the other sampling times.

Primarily, the decrease in %-macrophages was explained by an increase in %-neutrophils in all High Dose groups compared to Low Dose groups. However, the changes were similar to those observed in the oxidized ORP-100 control group. Lymphocyte and eosinophil differentials were variable among TA and dose groups with no apparent trend.

All animals survived to necropsy; gross pathology findings were generally few, minimal in severity and consisted primarily of minimal red discolorations of the lung. Common microscopic findings consisted of rare to few scattered infiltrations of mononuclear cells and eosinophils. These findings were observed in animals from all groups, were considered background and interpreted to have occurred prior to test article exposure or were an artifact of the sacrifice procedure.

Overall, there was no evidence of adverse test article effects in clinical observations, BALF chemistry, cell count, cell differential, lung tissue cytokines, macroscopic findings or microscopic lung alterations in Sprague Dawley rats exposed to oxidized ORP-100, and reduced ORP-100 or reduced ORP100S test article at target delivered doses of 10 mg/kg and 40 mg/kg, and examined at 1, 4 or 20 hours post exposure. However, this study utilized a sucrose/EDTA formulation which had been developed in order to confer the maximal degree of redox stability to thioredoxin when lyophilized in the reduced form (PCT WO 2006/090127), and a sucrose formulation effect was apparent as shown in FIG. 9, right. Neutrophil influx indicative of inflammation was induced in the oxidized ORP-100 control (lacking thioredoxin activity). Reduced ORP-100 was partially able to mitigate the formulation effect whereas reduced ORP100S abrogated neutrophil influx almost completely.

A vehicle-only group was not included in the present study design, and therefore, it was not possible to isolate the effect on BALF cell counts and cytokines of the sucrose/EDTA formulation by itself. However, when considered in context of intratracheal delivery (Rancourt, R. C., et al., 2007, Reduced thioredoxin increases proinflammatory cytokines and neutrophil influx in rat airways: modulation by airway mucus, Free Radic Biol Med 42, 1441-53), which indicated that an oxidized thioredoxin inactive protein control formulated in normal saline had identical BALF cell count and cytokine results as normal saline alone, the results of the present study (which showed the highest responses from the oxidized ORP-100 inactive protein control group) are consistent with the sucrose/EDTA formulation being responsible for observed dose-dependent inflammatory responses which were abrogated by reduced ORP100S.

The invention illustratively disclosed herein suitably may be practiced in the absence of any element which is not specifically disclosed herein. It is apparent to those skilled in the art, however, that many changes, variations, modifications, other uses, and applications of the invention are possible, and also changes, variations, modifications, other uses, and applications which do not depart from the spirit and scope of the invention are deemed to be covered by the invention, which is limited only by the claims which follow.

The foregoing discussion of the invention has been presented for purposes of illustration and description. The foregoing is not intended to limit the invention to the form or forms disclosed herein. In the foregoing Detailed Description of the Invention, for example, various features of the invention are grouped together in one or more embodiments for the purpose of streamlining the disclosure. The features of the embodiments of the invention may be combined in alternate embodiments other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description of the Invention, with each claim standing on its own as a separate preferred embodiment of the invention.

Moreover, though the description of the invention has included description of one or more embodiments and certain variations and modifications, other variations, combinations, and modifications are within the scope of the invention, e.g. as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, whether or not such alternate, interchangeable, and/or equivalent structures, functions, ranges, or steps are disclosed herein, and without intending to publicly dedicate any patentable subject matter.

1-72. (canceled)

73. A protein or peptide comprising the amino acid sequence of SEQ ID NO:28.

74. The protein or peptide of claim 73 comprising the amino acid sequence of SEQ ID NO:29.

75. The protein or peptide of claim 73 wherein the cysteine residue is in the reduced state.

76. A pharmaceutical composition comprising:
a) the protein or peptide of claim 73; and
b) a pharmaceutically acceptable excipient.

77. The pharmaceutical composition of claim 76, wherein the protein or peptide comprises the amino acid sequence of SEQ ID NO:29.

78. The pharmaceutical composition of claim 76 consisting essentially of:
a) the protein or peptide of claim 73;
b) water; and
c) sodium chloride.

79. The pharmaceutical composition of claim 76, wherein the pharmaceutically acceptable excipient is normal saline.

80. The pharmaceutical composition of claim 76, wherein the composition is a dry powder.

81. The pharmaceutical composition of claim 80, wherein the composition has a water content of less than about 3.0 wt. %.

82. The pharmaceutical composition of claim 76, wherein the protein or peptide is in a reduced state is operable to activate one or more endogenous antimicrobial peptides, wherein the activation results in a therapeutically effective reagent to treat or prevent infectious diseases.

83. The pharmaceutical composition of claim 76, wherein the composition does not include a thioredoxin protein fraction having UV absorbance greater than about 400 nm wavelength.

84. The pharmaceutical composition of claim 76, wherein said composition is formulated for administration to a patient by a route selected from the group consisting of oral, rectal, nasal, inhaled, intratracheal, bronchial, direct instillation, topical, and ocular.

85. A method for treating a disease or condition in a patient, comprising administering to said patient a pharmaceutical composition according to claim 76, wherein said disease or condition is selected from the group consisting of a disease associated with excessively viscous or cohesive mucus or sputum, inflammation, bacterial infection, a condition requiring modulation of the microbiome composition of said patient, and a viral respiratory disease.

86. The method of claim 85, wherein the protein or peptide has the amino acid sequence of SEQ ID NO:29.

87. The method of claim 85, wherein the inflammation is lung inflammation associated with a viral infection.

88. The method of claim 85, wherein the disease is selected from the group consisting of cystic fibrosis, chronic obstructive pulmonary disease, bronchiectasis, asthma, sinusitis, idiopathic pulmonary fibrosis, pulmonary hypertension, dry eye disease, and a digestive tract disease.

89. The method of claim 85, wherein the patient suffers from a disease associated with excessively viscous or cohesive mucus or sputum and the composition is contacted to the mucus or sputum of the patient by introducing the composition to the patient by a route selected from the group consisting of nasal administration, intratracheal administration, bronchial administration, direct installation into the lung, inhalation, oral administration, and ocular administration.

90. The method of claim 85, wherein the viral respiratory disease is selected from the group consisting of Acute Respiratory Distress Syndrome (ARDS), Severe Acute Respiratory Distress Syndrome (SARS), Middle East Respiratory Syndrome (MERS), SARS-Coronavirus-2 (SARS-CoV-19 or COVID-19), influenza, viral infection associated with asthma, pneumonia, bronchitis, tuberculosis, reactive airway disease syndrome, interstitial lung disease, a viral infection associated with a respiratory syncytial virus (RSV), a viral infection associated with a parainfluenza virus, and viral infection associated with a respiratory adenovirus.

91. A method of preparing a dried composition, comprising:
a) providing an aqueous composition comprising the protein or peptide of claim 73 and an aqueous solvent having a vapor pressure of at least about 3 mmHg; and
b) volatilizing the aqueous solvent to produce a dried composition comprising the protein or peptide.

92. The method of claim 91, wherein the protein or peptide has the amino acid sequence of SEQ ID NO:29.
